SEQUENCE LISTING (19 sequences)

SEQ ID NO: 1 - DNA, 45 bp, Artificial Sequence
  A synthetic oligonucleotide
gtcactatggcgtgctgctggatccatatgcgttgatgcaatttc

SEQ ID NO: 2 - DNA, 45 bp, Artificial Sequence
  A synthetic oligonucleotide
gaaattgcatcaacgcatatggatccagcagcacgccatagtgac

SEQ ID NO: 3 - DNA, 29 bp, Artificial Sequence
  A synthetic oligonucleotide
gcctggcttgcttccaaggaagccagcta

SEQ ID NO: 4 - DNA, 29 bp, Artificial Sequence
  A synthetic oligonucleotide
tagctggcttccttggaagcaagccaggc

SEQ ID NO: 5 - DNA, 36 bp, Artificial Sequence
  A synthetic oligonucleotide
cgcgcgttcgaacaaaatgtacccggatttaaaagg

SEQ ID NO: 6 - DNA, 32 bp, Artificial Sequence
  A synthetic oligonucleotide
gaattagaattcttaaccgcggcctgcctgga

SEQ ID NO: 7 - DNA, 36 bp, Artificial Sequence
  A synthetic oligonucleotide
gcgcgcttcgaacaaaatgccttctattaagttgaa

SEQ ID NO: 8 - DNA, 32 bp, Artificial Sequence
  A synthetic oligonucleotide
ggcgagcaattgttagacgaagataggaatct

SEQ ID NO: 9 - DNA, 23 bp, Artificial Sequence
  A synthetic oligonucleotide
atgtcnatnaanttnaantcngg

SEQ ID NO: 10 - DNA, 20 bp, Artificial Sequence
  A synthetic oligonucleotide
ctanacaaanantggaatgt

SEQ ID NO: 11 - DNA, 36 bp, Artificial Sequence
  A synthetic oligonucleotide
cgcggcttcgaacaaaatgtcgattaaattaaattc

SEQ ID NO: 12 - DNA, 36 bp, Artificial Sequence
  A synthetic oligonucleotide
taagctgaattcctagacaaagattggaatgtgatc

SEQ ID NO: 13 - DNA, 40 bp, Artificial Sequence
  A synthetic oligonucleotide
ctcaacctcgttgccgtagtcgcaggcaccatcgaagagg

SEQ ID NO: 14 - DNA, 40 bp, Artificial Sequence
  A synthetic oligonucleotide
cctcttcgatggtgcctgcgactacggcaacgaggttgag

SEQ ID NO: 15 - DNA, 27 bp, Artificial Sequence
  A synthetic oligonucleotide
gttggtgggctggttgaagcggatgcc

SEQ ID NO: 16 - DNA, 36 bp, Artificial Sequence
  A synthetic oligonucleotide
gcgcgcttcgaacaaaatggttcctgctatcaagct

SEQ ID NO: 17 - DNA, 41 bp, Artificial Sequence
  A synthetic oligonucleotide
aggttctcagcggagaagtagttggtgggctggttgaagcg

SEQ ID NO: 18 - DNA, 36 bp, Artificial Sequence
  A synthetic oligonucleotide
ctaaccgaaaatccagaggttctcagcggagaagta

SEQ ID NO: 19 - DNA, 30 bp, Artificial Sequence
  A synthetic oligonucleotide
acgcagtgaggggacaacatgagccgaagt

(19) United States
(12) Patent Application Publication
(10) Pub. No.: US 2011/0207190 A1
(43) Pub. Date: Aug. 25, 2011

(54) METHODS OF XYLITOL PREPARATION

(75) Inventors: Venkiteswaran Subramanian, Coralville, IA (US); Michael Tai-Man Louie, North Liberty, IA (US)

(73) Assignee: University of Iowa Research Foundation, Iowa City, IA (US)

(21) Appl. No.: 13/020,588

(22) Filed: Feb. 3, 2011

Related U.S. Application Data
(60) Provisional application No. 61/301,483, filed on Feb. 4, 2010.

(51) Int. Cl. C12P 7/18; C12N 1/19; C12N 1/21; C12N 9/04

(52) U.S. Cl. 435/158; 435/254.23; 435/254.21; 435/254.22; 435/254.2; 435/252.3; 435/190

(57) ABSTRACT

The invention provides spray-dried preparations of microbes useful for oxidoreductase reactions, e.g., xylitol production, and methods of making and using those microbes.

### CLAIM OF PRIORITY

This application claims the benefit of priority under 35 U.S.C. 119(e) from U.S. Provisional Application Ser. No. 61/301,483, filed Feb. 4, 2010, the disclosure of which is incorporated herein by reference in its entirety.

### BACKGROUND

Xylitol is a five-carbon sugar alcohol with an energy value of 2.4 cal/g compared to the 4 cal/g of sucrose and a sweetness comparable to sucrose. In contrast to sucrose, xylitol has beneficial health properties (Granstrom et al., 2007a; Granstrom et al., 2007b). It prevents dental caries and ear infection in small children and the metabolism of xylitol is insulin independent, and so it is an ideal sweetener for diabetics. Therefore, xylitol has become a global sweetener in toothpaste, chewing gums, and mints.

Xylitol is currently produced by chemical reduction, with a nickel catalyst, of D-xylose from birch wood hydrolysates (Granstrom et al., 2007a; Granstrom et al., 2007b). Recombinant DNA technology has allowed for the construction of microbial strains that are intended to serve as economic enzyme sources. For instance, metabolically engineered Saccharomyces cerevisiae and natural Candida sp. have been studied as an alternative for xylitol production. However, utilization of natural or metabolically engineered yeast strains to produce xylitol from biomass hydrolysate has several problems. Hydrolysates usually contain phenolic compounds and furfurals which are toxic and inhibit the growth of yeasts. Consequently, fermentation time is usually long. In addition, a portion of xylose in the hydrolysate is being used to produce biomass; hence xylitol yield is reduced. Since fermentation and catalysis happen concurrently, xylitol is produced simultaneously with other fermentation by-products such as acetate, lactate, and glycerol. This poses a problem in downstream purification of the product. Moreover, fermentation of xylose to xylitol takes a long time, about 4 to about 10 days.

### SUMMARY OF THE INVENTION

The invention provides a method to prepare a microbial cell biocatalyst. The method includes providing a microbial cell suspension, or a lysate or crude extract thereof, having an oxidoreductase, such as a NAD/NADH or NADP/NADPH-dependent oxidoreductase. In one embodiment, the oxidoreductase is one that is NADH-specific (e.g., one that has a specific activity with NADH that is at least 10 percent higher than with NADPH). In one embodiment, the oxidoreductase is one that is NADPH-specific (e.g., one that has a specific activity with NADPH that is at least 10 percent higher than with NADH). In one embodiment, the oxidoreductase is one that is not NADH-specific nor NADPH-specific, e.g., an oxidoreductase that is capable of utilizing both cofactors in the oxidized or reduced form. In one embodiment, the microbial cell is grown in media substantially lacking one or more inhibitors of the growth of the microbial cell. In one embodiment, microbial cells are grown to a very high cell density (e.g., A₆₀₀=about 200 to about 450 and wet weight of about 100 to about 300 g/L). The microbial cell suspension or a lysate or crude extract thereof, is then spray-dried under conditions effective to yield a spray-dried preparation, e.g., spray-dried cells, a spray-dried lysate of microbial cells or a spray-dried crude extract of microbial cells, suitable for biocatalysis. In one embodiment, the oxidoreductase is xylose reductase. In one embodiment, the oxidoreductase is sorbose reductase. In one embodiment, the oxidoreductase is mannitol dehydrogenase. In one embodiment, the oxidoreductase is a cytochrome P450 enzyme. In one embodiment, the oxidoreductase is a monooxygenase. In one embodiment, the oxidoreductase is a dioxygenase. In one embodiment, the oxidoreductase is phenylalanine dehydrogenase. In one embodiment, the oxidoreductase is a hydroxylase, e.g., 4-hydroxyphenylacetate 3-hydoxylase (HpaB and HpaC).

The invention thus provides a recombinant microbe, e.g., recombinant yeast, useful to produce a product, e.g., xylitol from a source having D-xylose, mannitol from a source having mannose, sorbitol from a source having L-sorbose, L-DOPA from a source having catechol, tyrosine or tyrosine and tetrahydrobiopterin, or in a screening assay, e.g., to detect metabolites of drugs, which employs one or more cytochrome P450 enzymes optionally in conjunction with non cytochrome P450 enzymes, e.g., a cytochrome P450 enzyme and glutathione S-transferase. The preparation of the recombinant microbe involves separating fermentation of the recombinant microbe from biotransformation by the oxidoreductase, e.g., of D-xylose to xylitol.

For example, a xylose reductase gene is cloned into a microbe, e.g., a yeast such as Pichia pastoris, a methylotrophic industrial yeast that does not grow on D-xylose (Iran and Meagher, 2001). In one embodiment, the recombinant microbe of the invention, e.g., recombinant yeast, expresses wild-type dihydroxyacetone synthase, e.g., the dihydroxyacetone synthase 2 gene is not mutated. In one embodiment, the xylose reductase is a heterologous xylose reductase. A heterologous dehydrogenase gene, such as one from Bacillus subtilis, may also be cloned into the same microbe, e.g., P. pastoris strain, to generate xylitol from xylose.

In one embodiment, a recombinant microbe such as a recombinant P. pastoris is then cultivated to high cell density in a defined medium under standardized conditions. Surprisingly, the introduction of a construct encoding a recombinant enzyme with relatively high activity in vitro into cells did not necessarily result in spray-dried cells with better activity. Moreover, it was also surprising that expression levels did not necessarily correlate with activity. For instance, as disclosed hereinbelow, the rate and percent conversion of xylose to xylitol by xylose reductase was independent of the level of expression of xylose reductase.

In one embodiment, during the fermentation phase, over-production of the exogenously introduced oxidoreductase is induced. After fermentation, the cells are spray-dried which renders the cells porous but the enzymes within the cells remain stable and are retained inside the spray-dried cells due to the pore size. The resulting spray-dried preparations of microbial cells such as bacterial and yeast cells, or lysates or crude extracts thereof, are suitable for biocatalysis, e.g., of complex mixtures including hemicellulose hydrosylates. For example, the pores of the spray-dried cells allow for substrates and products, such as xylose and xylitol, to diffuse freely in and out of the spray-dried cells. Spray drying results in enzymes that are stable at room temperature, and may permit processing in water rather than a buffer at a particular pH. The stability and lack of leaching of enzymes from spray-dried microbial cells allows for repeated use of the cells over time, e.g., continuous and extended use of a single preparation of spray-dried cells. In one embodiment, the spray-dried cells enable conversion of a substrate to a product in water or buffer or in a complex mixture such as a hemicellulose hydrolysates without the addition, e.g., of catalytic amounts, of an electron donor, e.g., conversion of xylose to xylitol in a hemicellulose hydroslyates without the addition of NAD(P)H or glucose or formate, for multiple rounds of conversion. The reaction times and conversion rates for one cycle with the spray-dried cells of the invention are rapid, e.g., from time of less than one hour up to a few hours, and high conversion, e.g., from 50 to 90% conversion, respectively, and result in stoichiometric conversion of substrates to products and a substantially pure product stream.

In one embodiment, the invention provides for spray-dried yeast preparations. In one embodiment, the spray-dried yeast cells contain heterologous xylose reductase. In one embodiment, spray-dried yeast cells have at least two recombinant enzymes. In one embodiment, spray-dried yeast cells suitable for xylitol production, such as those expressing xylose reductase and dehydrogenase, are provided. In one embodiment, the recombinant spray-dried yeast cells have xylose reductase and glucose dehydrogenase. For example, in one embodiment, spray-dried Pichia cells have Pichia stipitis or N. crassa xylose reductase and B. subtilis glucose dehydrogenase. The spray-dried cells are used for transformation of D-xylose in hemicellulose hydrolysate to xylitol in a biocatalytic phase without the addition of NAD(P)H or glucose or formate. However, the invention is not limited to particular microbes and/or particular sources of enzyme, as it is contemplated that microbes generally, whether recombinant or nonrecombinant, may be spray-dried and result in a source of stable oxidoreduxctase enzyme(s) for biocatalysis without the addition of electron donors, such as NAD(P)H or co-substrates such as glucose or formate. The reaction times and conversion rates for one cycle with the spray-dried cells of the invention are rapid, e.g., from time of less than one hour up to a few hours, and high, e.g., from 50 to 90%, respectively, and result in stoichiometric conversion of xylose to xylitol and a substantially pure product stream.

The use of spray-dried microbial cells may reduce the number of process steps for biocatalysis-based product production. For instance, the number of steps may be reduced by at least 2- to 3-fold using spray-dried microbial cells, thereby providing a simpler process and significant cost advantages. In one embodiment, a microbial cell suspension may be spray-dried directly from the fermentor, thus eliminating a solid/liquid separation step. For example, the cell containing solution directly from the fermentor (without separation) may be spray-dried or first subjected to cell separation and resuspension, e.g., in water or a buffer, prior to spray drying.

Other advantages of the method of the invention are that conditions and economics for high cell density fermentation of, for example, P. pastoris, are well-defined, and since a defined medium instead of biomass hydrolysate may be used in P. pastoris fermentation, there is no inhibition of growth by toxic components in a hydrolysate or other complex mixture. The spray-dried cells can be re-used in the multiple rounds of conversion of substrate to product with the potential to reduce the overall production cost. Also, as disclosed herein spray-dried cells used for xylitol production are not inhibited by potential toxic components in the hydrolysate or other complex mixture sources of xylose.

As described below, three xylose reductase genes from three different organisms were individually cloned into P. pastoris, creating 3 “single recombinant” strains. A Bacillus subtilis glucose dehydrogenase gene was also cloned into these 3 single recombinant P. pastoris to create 3 “double recombinant” strains. Expressions of xylose reductase and glucose dehydrogenase were confirmed by enzyme activity assays and SDS-PAGE analyses. The results further showed that double recombinant strains (e.g., P. pastoris with both xylose reductase and glucose dehydrogenase genes) and single recombinant strains (e.g., P. pastoris with xylose reductase alone) were capable of transforming D-xylose to xylitol. The double recombinant spray-dried cells were recycled for six rounds of biotransformation and remained active when an external source of NAD⁺ was present. Surprisingly, the reaction proceeded without the need of addition of electron donor like glucose or formate. The double recombinant spray-dried cells still remained active for 2 cycles without the addition of NAD⁺ and electron donor. The single recombinant spray-dried cells were capable of D-xylose to xylitol transformation without adding NAD⁺ to the reaction mixture. However, formate (an external electron donor) is needed to sustain the reaction for multiple cycles with the single recombinant. Both types of spray-dried cells were active in converting D-xylose in a hemicelluloses hydrolysate to xylitol. In one embodiment, the spray-dried cells produced 65 g/L xylitol from 90 g/L D-xylose in 2 hours. Conversion rates were at least 60%, e.g., at least 70%, including at least 75%, 80% or more.

The invention provides a method to prepare a variety of products via biocatalysis which employs a spray-dried microbial cell preparation expressing at least one recombinant enzyme which is optionally heterologous to the microbial cell and that together catalyze oxidoreductase reactions that produce the product. The reactions include those resulting in efficient conversions, e.g., conversions of at least 20% or more, e.g., at least 30%, 40% 50%, 60%, 70% or 80%. The biocatalysis may be conducted under aerobic conditions. In one embodiment, the biocatalytic method includes a spray-dried whole cell preparation of the invention and a mixture having xylose, e.g., hemicellulose hydrosylate such as one prepared from grasses, cereals, hardwoods or softwoods. In one embodiment, the method includes combining the spray-dried microbial cell biocatalyst with NADH or NADPH, e.g., the oxidized form of NADH, i.e., NAD⁺, which may reduce the cost of xylitol production. To provide for NADH, a dehydrogenase is also provided, e.g., by the whole microbial cell biocatalyst, to reduce NAD⁺ to NADH. In one embodiment, the NAD⁺ is reduced to NADH by formate dehydrogenase, e.g., in the presence of formate. In one embodiment, the NAD⁺ is reduced to NADH by glucose dehydrogenase, e.g., in the presence of glucose. In the absence of NAD⁺, microbial cells of the invention are reusable for at least two rounds. In one embodiment, the biocatalytic method does not include an exogenously added electron donor, e.g., does not include exogenously added formate or glucose, and/or an exogenously added electron carrier. Thus, the invention provides a method to prepare products of an oxidoreductase mediated reaction, such as xylitol. For instance, conversion rates for xylitol production were at least 60%, e.g., at least 70%, including at least 75%, 80% or more, over a period of time including but not limited to about 15 minutes up to about 10 hours, e.g., about 1 hour to about 4 hours, although lower conversion rates are envisioned for an oxidoreductase in a spray-dried preparation of the invention, for instance, conversion rates from at least 20%.

The method includes isolating the product from the aqueous medium of the biocatalyic reaction.

### BRIEF DESCRIPTION OF THE FIGURES

FIG. 1. Schematics of plasmids pPIC3.5K, pPIC3.5Kx, and pPIC4Kx. The Afe1 site (in red) in pPIC3.5K was mutated to a BamHI site in pPIC3.5Kx. Then, the Afe1-BstZ171 fragment in pPIC3.5Kx was removed, creating pPIC4Kx. Plasmid pPIC4Kx had a unique Bg/II site and a unique AsuII site (in blue).

FIG. 2. Procedure for construction of the double recombinant plasmid pPIC4Kx-PsXR-gdh from pPIC4Kx-PsXR and pPIC4Kx-gdh.

FIG. 3. SDS-PAGE gels loaded with cell extracts prepared from various single recombinant clones. The red arrows indicated the over-expressed XR protein. Lane C contained cell extracts of P. pastoris GS115 transformed with an empty pPIC4Kx plasmid without any cloned gene. Lanes 1 to 5: cell extracts of pPIC4Kx-PsXR clones 1000-1, 1000-2, 4000-1, 4000-2, and 4000-3, respectively. Lanes 6-10: cell extracts of pPIC4Kx-NcXR clones 1000-1, 1000-3, 4000-2, 4000-3, and 4000-6, respectively. Lanes 11-15: cell extracts of pPIC4Kx-CpXr clones 4000-8, 4000-6, 4000-2, 1000-6, and 1000-2, respectively.

FIG. 4. SDS-PAGE gels loaded with cell extracts prepared from various double recombinant clones. The red arrows indicated the over-expressed XR protein. Lane C contained cell extracts of P. pastoris GS 115 transformed with an empty pPIC4Kx plasmid without any cloned gene. Lanes 1 to 5: cell extracts of pPIC4Kx-gdh-NcXR clones 1000-1, 1000-2, 4000-1, 4000-2, and 4000-3, respectively. Lanes 6-10: cell extracts of pPIC4Kx-PsXR-gdh clones 1000-7, 1000-8, 4000-4, 4000-7, and 4000-8, respectively. Lanes 11-15: cell extracts of pPIC4Kx-CpXr-gdh clones 1000-5, 1000-6, 4000-4, 4000-5, and 4000-6, respectively.

FIG. 5. Biotransformation of D-xylose to xylitol by cell extracts of double recombinant clones NcXR+GDH 4000-1 and PsXR+GDH 4000-4 in the presence of NADPH.

FIG. 6. Biotransformation of D-xylose to xylitol by cell extracts of (A) NcXR+GDH 1000-2, (B) NcXR+GDH 4000-1, and (C) PsXR+GDH 4000-4. The reactions in (A) and (B) contained 13 and 24 U of NcXR activity, respectively. Reaction in (C) had 5.4 U of PsXR activity. At 0 hours (black), 2 hours (blue), and 12 hours (red), samples were drawn from each reaction for HPLC analyses.

FIG. 7. Biotransformation of D-xylose to xylitol by cell extracts of NcXR+GDH 1000-2. Reaction set up was identical to that described in FIG. 6, except 130 U of NcXR activity was used.

FIG. 8. Biotransformation of D-xylose to xylitol by cell extracts of (A and B) PsXR+GDH 4000-4 and (C and D) NcXR+GDH 4000-1. Reactions in (A and B) had 2.7 U of PsXR activity while reactions in (C and D) had 12 U of NcXR activity. Formate was present only in reactions in (A and C).

FIG. 9. Xylitol production using spray-dried (A) NcXR+GDH 4000-1 and (B) PsXR+GDH 4000-4 cells. Solid lines represent xylitol production while dashed lines represent D-xylose consumption.

FIG. 10. Xylitol production using spray-dried (A and C) PsXR+GDH 4000-4 and (B) NcXR+GDH 4000-1 cells. Reaction solutions in (A and B) contained 0.25 mM NAD⁺. Reaction solution in (C) had no NAD⁺ until NAD⁺ was added at the indicated time points (pink and black arrows).

FIG. 11. Xylitol production by spray-dried PsXR+GDH 4000-4 cells with repetitive addition of D-xylose (▴=D-xylose; Δ=xylitol). The 5-mL reaction contained 50 mg spray-dried cells in 50 mM KPi (pH 7.0) buffer without NAD⁺. Once production of xylitol stopped, an aliquot of D-xylose solution (2 M stock) was added to the reaction (represent by the black arrows), increasing the final D-xylose concentration by 200 mM.

FIG. 12. Biotransformation of D-xylose to xylitol by spray-dried single recombinant PsXR 4000-3 cells. The reaction mixture did not contain NADH at time=0 hours. NADH (50 mM) was added to the reaction 1.5 hours after the start of the reaction.

FIG. 13. Biotransformation of D-xylose to xylitol by spray-dried single recombinant (A) PsXR 1000-1, (B) PsXR 4000-3, and (C) NcXR 1000-1 cells. The reaction mixtures contained 200 mM formate but neither NAD⁺ nor NADH.

FIG. 14. HPLC analysis of the first batch of hemicelluloses hydrolysate. The black solid trace in the figure is the HPLC chromatogram of the hydrolysate. HPLC chromatograms of the various standard solutions are overlaid onto the hydrolysate chromatogram.

FIG. 15. Biotransformation of D-xylose in hemicelluloses hydrolysate by spray-dried PsXR+GDH 4000-4 cells. The dashed black trace represents the chromatogram of the hydrolysate in the absence of spray-dried cells. No xylitol peak was present in that sample. Once spray-dried cells were incubated with the hydrolysate, xylitol was produced immediately (solid black trace) and resulted in a small xylitol peak in the t=0 hour sample.

FIG. 16. ESI-MS of (A) xylitol produced from hemicelluloses hydrolysate by PsXR+GDH 4000-4 cells, (B) xylose standard, and (C) xylitol standard.

FIG. 17. HPLC analysis of the second batch of hemicelluloses hydrolysate after adjusting the pH of the solution to 7.0.

FIG. 18. Biotransformation of D-xylose in a hydrolysate solution to xylitol by spray-dried cells. (A to C) PsXR+GDH 4000-4; (D to F) PsXR 4000-3.

FIG. 19. Biotransformation of indole by 10 mg/mL spray-dried E. coli JM109(DE3) pDTG141 (sample in buffer) in a reaction mixture containing (A) control (boiled cells); (B) 50 mM buffer; (C) 250 μM NAD; (D) 200 μM NADH; and (E) 10 mM glucose. E. coli JM109(DE3) pDTG141 expresses Pseudomonas sp. strain NCIB 9816-4 naphthalene dioxygenase (NDO) from a T7 promoter.

### DETAILED DESCRIPTION OF THE INVENTION

### Definitions

The term “nucleic acid molecule”, “polynucleotide”, or “nucleic acid sequence” as used herein, refers to nucleic acid, DNA or RNA, that comprises coding sequences necessary for the production of a polypeptide or protein precursor. The encoded polypeptide may be a full-length polypeptide, a fragment thereof (less than full-length), or a fusion of either the full-length polypeptide or fragment thereof with another polypeptide, yielding a fusion polypeptide.

A “nucleic acid”, as used herein, is a covalently linked sequence of nucleotides in which the 3′ position of the pentose of one nucleotide is joined by a phosphodiester group to the 5′ position of the pentose of the next, and in which the nucleotide residues (bases) are linked in specific sequence, i.e., a linear order of nucleotides. A “polynucleotide”, as used herein, is a nucleic acid containing a sequence that is greater than about 100 nucleotides in length. An “oligonucleotide” or “primer”, as used herein, is a short polynucleotide or a portion of a polynucleotide. An oligonucleotide typically contains a sequence of about two to about one hundred bases. The word “oligo” is sometimes used in place of the word “oligonucleotide”.

Nucleic acid molecules are said to have a “5′-terminus” (5′ end) and a “3′-terminus” (3′ end) because nucleic acid phosphodiester linkages occur to the 5′ carbon and 3′ carbon of the pentose ring of the substituent mononucleotides. The end of a polynucleotide at which a new linkage would be to a 5′ carbon is its 5′ terminal nucleotide. The end of a polynucleotide at which a new linkage would be to a 3′ carbon is its 3′ terminal nucleotide. A terminal nucleotide, as used herein, is the nucleotide at the end position of the 3′- or 5′-terminus

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotides referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring.

As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. Typically, promoter and enhancer elements that direct transcription of a linked gene (e.g., open reading frame or coding region) are generally located 5′ or upstream of the coding region. However, enhancer elements can exert their effect even when located 3′ of the promoter element and the coding region. Transcription termination and polyadenylation signals are located 3′ or downstream of the coding region.

The term “codon” as used herein, is a basic genetic coding unit, consisting of a sequence of three nucleotides that specify a particular amino acid to be incorporated into a polypeptide chain, or a start or stop signal. The term “coding region” when used in reference to structural gene refers to the nucleotide sequences that encode the amino acids found in the nascent polypeptide as a result of translation of a mRNA molecule. Typically, the coding region is bounded on the 5′ side by the nucleotide triplet “ATG” which encodes the initiator methionine and on the 3′ side by a stop codon (e.g., TAA, TAG, TGA). In some cases the coding region is also known to initiate by a nucleotide triplet “TTG”.

The term “gene” refers to a DNA sequence that comprises coding sequences and optionally control sequences necessary for the production of a polypeptide from the DNA sequence.

As used herein, the term “heterologous” nucleic acid sequence or protein refers to a sequence that relative to a reference sequence has a different source, e.g., originates from a foreign species, or, if from the same species, it may be substantially modified from the original form.

Nucleic acids are known to contain different types of mutations. A “point” mutation refers to an alteration in the sequence of a nucleotide at a single base position from the wild-type sequence. Mutations may also refer to insertion or deletion of one or more bases, so that the nucleic acid sequence differs from a reference, e.g., a wild-type, sequence.

As used herein, the terms “hybridize” and “hybridization” refer to the annealing of a complementary sequence to the target nucleic acid, i.e., the ability of two polymers of nucleic acid (polynucleotides) containing complementary sequences to anneal through base pairing. The terms “annealed” and “hybridized” are used interchangeably throughout, and are intended to encompass any specific and reproducible interaction between a complementary sequence and a target nucleic acid, including binding of regions having only partial complementarity. Certain bases not commonly found in natural nucleic acids may be included in the nucleic acids of the present invention and include, for example, inosine and 7-deazaguanine. Those skilled in the art of nucleic acid technology can determine duplex stability empirically considering a number of variables including, for example, the length of the complementary sequence, base composition and sequence of the oligonucleotide, ionic strength and incidence of mismatched base pairs. The stability of a nucleic acid duplex is measured by the melting temperature, or “Tₘ”. The Tₘ of a particular nucleic acid duplex under specified conditions is the temperature at which on average half of the base pairs have disassociated.

The term “vector” is used in reference to nucleic acid molecules into which fragments of DNA may be inserted or cloned and can be used to transfer DNA segment(s) into a cell and capable of replication in a cell. Vectors may be derived from plasmids, bacteriophages, viruses, cosmids, and the like.

The terms “recombinant vector” and “expression vector” as used herein refer to DNA or RNA sequences containing a desired coding sequence and appropriate DNA or RNA sequences necessary for the expression of the operably linked coding sequence in a particular host organism. Prokaryotic expression vectors include a promoter, a ribosome binding site, an origin of replication for autonomous replication in a host cell and possibly other sequences, e.g. an optional operator sequence, optional restriction enzyme sites. A promoter is defined as a DNA sequence that directs RNA polymerase to bind to DNA and to initiate RNA synthesis, which RNA in eukaryotes may be processed to mRNA. Eukaryotic expression vectors include a promoter, optionally a polyadenylation signal and optionally an enhancer sequence.

A polynucleotide having a nucleotide sequence encoding a protein or polypeptide means a nucleic acid sequence comprising the coding region of a gene, or in other words the nucleic acid sequence encodes a gene product. The coding region may be present in either a cDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. In further embodiments, the coding region may contain a combination of both endogenous and exogenous control elements.

The term “transcription regulatory element” or “transcription regulatory sequence” refers to a genetic element or sequence that controls some aspect of the expression of nucleic acid sequence(s). For example, a promoter is a regulatory element that facilitates the initiation of transcription of an operably linked coding region. Other regulatory elements include, but are not limited to, transcription factor binding sites, splicing signals, polyadenylation signals, termination signals and enhancer elements.

Transcriptional control signals in eukaryotes comprise “promoter” and “enhancer” elements. Promoters and enhancers consist of short arrays of DNA sequences that interact specifically with cellular proteins involved in transcription. Promoter and enhancer elements have been isolated from a variety of eukaryotic sources including genes in yeast, insect and mammalian cells. Promoter and enhancer elements have also been isolated from viruses and analogous control elements, such as promoters, are also found in prokaryotes. The selection of a particular promoter and enhancer depends on the cell type used to express the protein of interest. Some eukaryotic promoters and enhancers have a broad host range while others are functional in a limited subset of cell types.

The term “promoter/enhancer” denotes a segment of DNA containing sequences capable of providing both promoter and enhancer functions (i.e., the functions provided by a promoter element and an enhancer element as described above). For example, the long terminal repeats of retroviruses contain both promoter and enhancer functions. The enhancer/promoter may be “endogenous” or “exogenous” or “heterologous.” An “endogenous” enhancer/promoter is one that is naturally linked with a given gene in the genome. An “exogenous” or “heterologous” enhancer/promoter is one that is placed in juxtaposition to a gene by means of genetic manipulation (i.e., molecular biological techniques) such that transcription of the gene is directed by the linked enhancer/promoter.

The presence of “splicing signals” on an expression vector often results in higher levels of expression of the recombinant transcript in eukaryotic host cells. Splicing signals mediate the removal of introns from the primary RNA transcript and consist of a splice donor and acceptor site. A commonly used splice donor and acceptor site is the splice junction from the 16S RNA of SV40.

Efficient expression of recombinant DNA sequences in eukaryotic cells requires expression of signals directing the efficient termination and polyadenylation of the resulting transcript. Transcription termination signals are generally found downstream of the polyadenylation signal and are a few hundred nucleotides in length. The term “poly(A) site” or “poly(A) sequence” as used herein denotes a DNA sequence which directs both the termination and polyadenylation of the nascent RNA transcript. Efficient polyadenylation of the recombinant transcript is desirable, as transcripts lacking a poly(A) tail are unstable and are rapidly degraded. The poly(A) signal utilized in an expression vector may be “heterologous” or “endogenous.” An endogenous poly(A) signal is one that is found naturally at the 3′ end of the coding region of a given gene in the genome. A heterologous poly(A) signal is one which has been isolated from one gene and positioned 3′ to another gene. A commonly used heterologous poly(A) signal is the SV40 poly(A) signal. The SV40 poly(A) signal is contained on a 237 by BamHI/BclI restriction fragment and directs both termination and polyadenylation.

The term “in vitro” refers to an artificial environment and to processes or reactions that occur within an artificial environment. In vitro environments include, but are not limited to, test tubes and cell lysates. The term “in vivo” refers to the natural environment (e.g., an animal or a cell) and to processes or reaction that occur within a natural environment.

The term “expression system” refers to any assay or system for determining (e.g., detecting) the expression of a gene of interest. Those skilled in the field of molecular biology will understand that any of a wide variety of expression systems may be used. The method of transformation or transfection and the choice of expression vehicle will depend on the host system selected. Transformation and transfection methods are well known to the art. Expression systems include in vitro gene expression assays where a gene of interest (e.g., a reporter gene) is linked to a regulatory sequence and the expression of the gene is monitored following treatment with an agent that inhibits or induces expression of the gene. Detection of gene expression can be through any suitable means including, but not limited to, detection of expressed mRNA or protein (e.g., a detectable product of a reporter gene) or through a detectable change in the phenotype of a cell expressing the gene of interest. Expression systems may also comprise assays where a cleavage event or other nucleic acid or cellular change is detected.

The term “wild-type” as used herein, refers to a gene or gene product that has the characteristics of that gene or gene product isolated from a naturally occurring source. A wild-type gene is that which is most frequently observed in a population and is thus arbitrarily designated the “wild-type” form of the gene. In contrast, the term “mutant” refers to a gene or gene product that displays modifications in sequence and/or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “isolated” when used in relation to a nucleic acid, as in “isolated oligonucleotide” or “isolated polynucleotide” refers to a nucleic acid sequence that is identified and separated from at least one contaminant with which it is ordinarily associated in its source. Thus, an isolated nucleic acid is present in a form or setting that is different from that in which it is found in nature. In contrast, non-isolated nucleic acids (e.g., DNA and RNA) are found in the state they exist in nature. For example, a given DNA sequence (e.g., a gene) is found on the host cell chromosome in proximity to neighboring genes; RNA sequences (e.g., a specific mRNA sequence encoding a specific protein), are found in the cell as a mixture with numerous other mRNAs that encode a multitude of proteins. However, isolated nucleic acid includes, by way of example, such nucleic acid in cells ordinarily expressing that nucleic acid where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide contains at a minimum, the sense or coding strand (i.e., the oligonucleotide may single-stranded), but may contain both the sense and anti-sense strands (i.e., the oligonucleotide may be double-stranded).

By “peptide,” “protein” and “polypeptide” is meant any chain of amino acids, regardless of length or post-translational modification (e.g., glycosylation or phosphorylation). The nucleic acid molecules of the invention may also encode a variant of a naturally-occurring protein or polypeptide fragment thereof, which has an amino acid sequence that is at least 85%, 90%, 95% or 99% identical to the amino acid sequence of the naturally-occurring (native or wild-type) protein from which it is derived. The term “fusion polypeptide” or “fusion protein” refers to a chimeric protein containing a reference protein (e.g., luciferase) joined at the N- and/or C-terminus to one or more heterologous sequences (e.g., a non-luciferase polypeptide). In some embodiments, a modified polypeptide, fusion polypeptide or a portion of a full-length polypeptide of the invention, may retain at least some of the activity of a corresponding full-length functional (nonchimeric) polypeptide. In other embodiments, in the absence of an exogenous agent or molecule of interest, a modified polypeptide, fusion polypeptide or portion of a full-length functional polypeptide of the invention, may lack activity relative to a corresponding full-length functional polypeptide. In other embodiments, a modified polypeptide, fusion polypeptide or portion of a full-length functional polypeptide of the invention in the presence of an exogenous agent may retain at least some or have substantially the same activity, or alternatively lack activity, relative to a corresponding full-length functional polypeptide.

Polypeptide molecules are said to have an “amino terminus” (N-terminus) and a “carboxy terminus” (C-terminus) because peptide linkages occur between the backbone carboxyl group of a first amino acid residue and the backbone amino group of a second amino acid residue. The terms “N-terminal” and “C-terminal” in reference to polypeptide sequences refer to regions of polypeptides including portions of the N-terminal and C-terminal regions of the polypeptide, respectively. A sequence that includes a portion of the N-terminal region of polypeptide includes amino acids predominantly from the N-terminal half of the polypeptide chain, but is not limited to such sequences. For example, an N-terminal sequence may include an interior portion of the polypeptide sequence including bases from both the N-terminal and C-terminal halves of the polypeptide. The same applies to C-terminal regions. N-terminal and C-terminal regions may, but need not, include the amino acid defining the ultimate N-terminus and C-terminus of the polypeptide, respectively.

The term “recombinant protein” or “recombinant polypeptide” as used herein refers to a protein molecule expressed from a recombinant DNA molecule. In contrast, the term “native protein” is used herein to indicate a protein isolated from a naturally occurring (i.e., a nonrecombinant) source. Molecular biological techniques may be used to produce a recombinant form of a protein with identical properties as compared to the native form of the protein.

The terms “cell,” “cell line,” “host cell,” as used herein, are used interchangeably, and all such designations include progeny or potential progeny of these designations. By “transformed cell” is meant a cell into which (or into an ancestor of which) has been introduced a nucleic acid molecule of the invention. Optionally, a nucleic acid molecule of the invention may be introduced into a suitable cell line so as to create a stably-transfected cell line capable of producing the protein or polypeptide encoded by the gene. Vectors, cells, and methods for constructing such cell lines are well known in the art. The words “transformants” or “transformed cells” include the primary transformed cells derived from the originally transformed cell without regard to the number of transfers. All progeny may not be precisely identical in DNA content, due to deliberate or inadvertent mutations. Nonetheless, mutant progeny that have the same functionality as screened for in the originally transformed cell are included in the definition of transformants.

The term “homology” refers to a degree of complementarity between two or more sequences. There may be partial homology or complete homology (i.e., identity). Homology is often measured using sequence analysis software (e.g., Sequence Analysis Software Package of the Genetics Computer Group. University of Wisconsin Biotechnology Center. 1710 University Avenue. Madison, Wis. 53705). Such software matches similar sequences by assigning degrees of homology to various substitutions, deletions, insertions, and other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

The term “isolated” when used in relation to a polypeptide, as in “isolated protein” or “isolated polypeptide” refers to a polypeptide that is identified and separated from at least one contaminant with which it is ordinarily associated in its source. Thus, an isolated polypeptide is present in a form or setting that is different from that in which it is found in nature. In contrast, non-isolated polypeptides (e.g., proteins and enzymes) are found in the state they exist in nature.

The term “purified” or “to purify” means the result of any process that removes some of a contaminant from the component of interest, such as a protein or nucleic acid. The percent of a purified component is thereby increased in the sample.

As used herein, “pure” means an object species is the predominant species present (i.e., on a molar basis it is more abundant than any other individual species in the composition), and optionally a substantially purified fraction is a composition wherein the object species comprises at least about 50 percent (on a molar basis) of all macromolecular species present. Generally, a “substantially pure” composition will comprise more than about 80 percent of all macromolecular species present in the composition, for example, more than about 85%, about 90%, about 95%, and about 99%. In one embodiment, the object species is purified to essential homogeneity (contaminant species cannot be detected in the composition by conventional detection methods) wherein the composition consists essentially of a single macromolecular species.

The term “operably linked” as used herein refer to the linkage of nucleic acid sequences in such a manner that a nucleic acid molecule capable of directing the transcription of a given gene and/or the synthesis of a desired protein molecule is produced. The term also refers to the linkage of sequences encoding amino acids in such a manner that a functional (e.g., enzymatically active, capable of binding to a binding partner, capable of inhibiting, etc.) protein or polypeptide is produced.

As used herein, a “marker gene” or “reporter gene” is a gene that imparts a distinct phenotype to cells expressing the gene and thus permits cells having the gene to be distinguished from cells that do not have the gene. Such genes may encode either a selectable or screenable marker, depending on whether the marker confers a trait which one can ‘select’ for by chemical means, i.e., through the use of a selective agent (e.g., a herbicide, antibiotic, or the like), or whether it is simply a “reporter” trait that one can identify through observation or testing, i.e., by ‘screening’. Elements of the present disclosure are exemplified in detail through the use of particular marker genes. Of course, many examples of suitable marker genes or reporter genes are known to the art and can be employed in the practice of the invention. Therefore, it will be understood that the following discussion is exemplary rather than exhaustive. In light of the techniques disclosed herein and the general recombinant techniques which are known in the art, the present invention renders possible the alteration of any gene. Exemplary modified reporter proteins are encoded by nucleic acid molecules comprising modified reporter genes including, but are not limited to, modifications of a neo gene, a β-gal gene, a gus gene, a cat gene, a gpt gene, a hyg gene, a hisD gene, a ble gene, a mprt gene, a bar gene, a nitrilase gene, a galactopyranoside gene, a xylosidase gene, a thymidine kinase gene, an arabinosidase gene, a mutant acetolactate synthase gene (ALS) or acetoacid synthase gene (AAS), a methotrexate-resistant dhfr gene, a dalapon dehalogenase gene, a mutated anthranilate synthase gene that confers resistance to 5-methyl tryptophan (WO 97/26366), an R-locus gene, a β-lactamase gene, a xy/E gene, an α-amylase gene, a tyrosinase gene, a luciferase (luc) gene, (e.g., a Renilla reniformis luciferase gene, a firefly luciferase gene, or a click beetle luciferase (Pyrophorus plagiophthalamus) gene), an aequorin gene, a red fluorescent protein gene, or a green fluorescent protein gene.

All amino acid residues identified herein are in the natural L-configuration. In keeping with standard polypeptide nomenclature, abbreviations for amino acid residues are as shown in the following Table of Correspondence.

TABLE OF CORRESPONDENCE

| 1-Letter | 3-Letter | AMINO ACID |
|---|---|---|
| Y | Tyr | L-tyrosine |
| G | Gly | L-glycine |
| F | Phe | L-phenylalanine |
| M | Met | L-methionine |
| A | Ala | L-alanine |
| S | Ser | L-serine |
| I | Ile | L-isoleucine |
| L | Leu | L-leucine |
| T | Thr | L-threonine |
| V | Val | L-valine |
| P | Pro | L-proline |
| K | Lys | L-lysine |
| H | His | L-histidine |
| Q | Gln | L-glutamine |
| E | Glu | L-glutamic acid |
| W | Trp | L-tryptophan |
| R | Arg | L-arginine |
| D | Asp | L-aspartic acid |
| N | Asn | L-asparagine |
| C | Cys | L-cysteine |

### Sources of Cells for Spray-Drying and Methods of Preparation and Use

The invention provides spray-dried preparations of microbial cells, or lysates or crude extracts thereof, suitable for biocatalysis, and a simpler process for using those cells, lysates or crude extracts thereof, in biocatalysis. In one embodiment of the invention, the invention provides for spray-dried preparations of prokaryotic cells, or lysates or crude extracts thereof, suitable for biocatalysis. In one embodiment, the prokaryotic cells are E. coli cells. In another embodiment, the prokaryotic cells are Pseudomonas cells. In another embodiment, the cells are eukaryotic cells, e.g., yeast cells, or lysates or crude extracts thereof, suitable for biocatalysis.

The present invention provides for a process to spray-dry microbial cells to render them porous and suitable for biocatalysis, without leaching of enzymes for the biocatalysis. Thus, the cells can be used directly for production. Accordingly, the process to take a biocatalyst from the fermentor to the reactor has been simplified by several steps. Spray-drying the cells may also render the enzymes in those cells stable. As described herein for recombinant cells with xylose reductase, or xylose reductase and a dehydrogenase, spray drying resulted in stable enzymes and a rapid rate of xylitol production. Accordingly, in one embodiment, the invention provides recombinant microbial cells, such as recombinant Pichia, Saccharomyces, Pseudomonas or E. coli cells, for the production of xylitol. The spray-dried cells are easy to prepare, store and use.

Yeast cells useful in the present invention are those from phylum Ascomycota, subphylum Saccharomycotina, class Saccharomycetes, order Saccharomycetales or Schizosaccharomycetales, family Saccharomycetaceae, genus Saccharomyces or Pichia (Hansenula), e.g., species: P. anomola, P. guilliermondiii, P. norvegenesis, P. ohmeri, and P. pastoris. Yeast cells employed in the invention may be native (non-recombinant) cells or recombinant cells, e.g., those which are transformed with exogenous (recombinant) DNA having one or more expression cassettes each with a polynucleotide having a promoter and an open reading frame encoding one or more enzymes useful for biocatalysis. The enzyme(s) encoded by the exogenous DNA is referred to as “recombinant,” and that enzyme may be from the same species or heterologous (from a different species). For example, a recombinant P. pastoris cell may recombinantly express a P. pastoris enzyme or a plant, microbial, e.g., Aspergillus or Saccharomyces, or mammalian enzyme.

In one embodiment, the microbial cell employed in the methods of the invention is transformed with recombinant DNA, e.g., in a vector. Vectors, plasmids, cosmids, YACs (yeast artificial chromosomes) BACs (bacterial artificial chromosomes) and DNA segments for use in transforming cells will generally comprise DNA encoding an enzyme, as well as other DNA that one desires to introduce into the cells. These DNA constructs can further include elements such as promoters, enhancers, polylinkers, marker or selectable genes, or even regulatory genes, as desired. For instance, one of the DNA segments or genes chosen for cellular introduction will often encode a protein that will be expressed in the resultant transformed (recombinant) cells, such as to result in a screenable or selectable trait and/or that will impart an improved phenotype to the transformed cell. However, this may not always be the case, and the present invention also encompasses transformed cells incorporating non-expressed transgenes.

DNA useful for introduction into cells includes that which has been derived or isolated from any source, that may be subsequently characterized as to structure, size and/or function, chemically altered, and later introduced into cells. An example of DNA “derived” from a source, would be a DNA sequence that is identified as a useful fragment within a given organism, and that is then chemically synthesized in essentially pure form. An example of such DNA “isolated” from a source would be a useful DNA sequence that is excised or removed from said source by biochemical means, e.g., enzymatically, such as by the use of restriction endonucleases, so that it can be further manipulated, e.g., amplified, for use in the invention, by the methodology of genetic engineering. Such DNA is commonly also referred to as “recombinant DNA.”

Therefore, useful DNA includes completely synthetic DNA, semi-synthetic DNA, DNA isolated from biological sources, and DNA derived from introduced RNA. The introduced DNA may be or may not be a DNA originally resident in the host cell genotype that is the recipient of the DNA (native or heterologous). It is within the scope of the invention to isolate a gene from a given genotype, and to subsequently introduce multiple copies of the gene into the same genotype, e.g., to enhance production of a given gene product.

The introduced DNA includes, but is not limited to, DNA from genes such as those from bacteria, yeasts, fungi, plants or vertebrates, e.g., mammals. The introduced DNA can include modified or synthetic genes, e.g., “evolved” genes, portions of genes, or chimeric genes, including genes from the same or different genotype. The term “chimeric gene” or “chimeric DNA” is defined as a gene or DNA sequence or segment comprising at least two DNA sequences or segments from species that do not combine DNA under natural conditions, or which DNA sequences or segments are positioned or linked in a manner that does not normally occur in the native genome of the untransformed cell.

The introduced DNA used for transformation herein may be circular or linear, double-stranded or single-stranded. Generally, the DNA is in the form of chimeric DNA, such as plasmid DNA, which can also contain coding regions flanked by regulatory sequences that promote the expression of the recombinant DNA present in the transformed cell. For example, the DNA may include a promoter that is active in a cell that is derived from a source other than that cell, or may utilize a promoter already present in the cell that is the transformation target.

Generally, the introduced DNA will be relatively small, i.e., less than about 30 kb to minimize any susceptibility to physical, chemical, or enzymatic degradation that is known to increase as the size of the DNA increases. The number of proteins, RNA transcripts or mixtures thereof that is introduced into the cell may be preselected and defined, e.g., from one to about 5-10 such products of the introduced DNA may be formed.

The selection of an appropriate expression vector will depend upon the host cells. An expression vector can contain, for example, (1) prokaryotic DNA elements coding for a bacterial origin of replication and an antibiotic resistance gene to provide for the amplification and selection of the expression vector in a bacterial host; (2) DNA elements that control initiation of transcription such as a promoter; (3) DNA elements that control the processing of transcripts such as introns, transcription termination/polyadenylation sequence; and (4) a gene of interest that is operatively linked to the DNA elements to control transcription initiation. The expression vector used may be one capable of autonomously replicating in the host cell or capable of integrating into the chromosome, originally containing a promoter at a site enabling transcription of the linked gene.

Yeast or fungal expression vectors may comprise an origin of replication, a suitable promoter and enhancer, and also any necessary ribosome binding sites, polyadenylation site, splice donor and acceptor sites, transcriptional termination sequences, and 5=flanking nontranscribed sequences. Several well-characterized yeast expression systems are known in the art and described in, e.g., U.S. Pat. No. 4,446,235, and European Patent Applications 103,409 and 100,561. A large variety of shuttle vectors with yeast promoters are also known to the art. However, any other plasmid or vector may be used as long as they are viable in the host. In one embodiment, the plasmid or vector is maintained extrachromosomally in the host cell. In one embodiment, the plasmid or vector is integrated into the chromosome of the host cell.

The construction of vectors that may be employed in conjunction with the present invention will be known to those of skill of the art in light of the present disclosure (see, e.g., Sambrook and Russell, Molecular Biology: A Laboratory Manual, 2001). The expression cassette of the invention may contain one or a plurality of restriction sites allowing for placement of the polynucleotide encoding an enzyme. The expression cassette may also contain a termination signal operably linked to the polynucleotide as well as regulatory sequences required for proper translation of the polynucleotide. The expression cassette containing the polynucleotide of the invention may be chimeric, meaning that at least one of its components is heterologous with respect to at least one of the other components. Expression of the polynucleotide in the expression cassette may be under the control of a constitutive promoter, inducible promoter, regulated promoter, viral promoter or synthetic promoter.

The expression cassette may include, in the 5′-3′ direction of transcription, a transcriptional and translational initiation region, the polynucleotide of the invention and a transcriptional and translational termination region functional in vivo and/or in vitro. The termination region may be native with the transcriptional initiation region, may be native with the polynucleotide, or may be derived from another source. The regulatory sequences may be located upstream (5′ non-coding sequences), within (intron), or downstream (3′ non-coding sequences) of a coding sequence, and influence the transcription, RNA processing or stability, and/or translation of the associated coding sequence. Regulatory sequences may include, but are not limited to, enhancers, promoters, repressor binding sites, translation leader sequences, introns, and polyadenylation signal sequences. They may include natural and synthetic sequences as well as sequences that may be a combination of synthetic and natural sequences.

The vector used in the present invention may also include appropriate sequences for amplifying expression.

A promoter is a nucleotide sequence that controls the expression of a coding sequence by providing the recognition for RNA polymerase and other factors required for proper transcription. A promoter includes a minimal promoter, consisting only of all basal elements needed for transcription initiation, such as a TATA-box and/or initiator that is a short DNA sequence comprised of a TATA-box and other sequences that serve to specify the site of transcription initiation, to which regulatory elements are added for control of expression. A promoter may be derived entirely from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions. A promoter may also include a minimal promoter plus a regulatory element or elements capable of controlling the expression of a coding sequence or functional RNA. This type of promoter sequence contains of proximal and more distal elements, the latter elements are often referred to as enhancers.

Representative examples of promoters include, but are not limited to, promoters known to control expression of genes in prokaryotic or eukaryotic cells or their viruses. For instance, any promoter capable of expressing in yeast hosts can be used as a promoter in the present invention, for example, the AOX (alcohol oxidase) gene promoter, e.g., the AOX1 or AOX2 promoter, may be used. Additional promoters useful for expression in a yeast cell are well described in the art. Examples thereof include promoters of the genes coding for glycolytic enzymes, such as TDH3, glyceraldehyde-3-phosphate dehydrogenase (GAPDH), a shortened version of GAPDH (GAPFL), 3-phosphoglycerate kinase (PGK), hexokinase, pyruvate decarboxylase, phosphofructokinase, glucose-6-phosphate isomerase, 3-phosphoglycerate mutase, pyruvate kinase, triosephosphate isomerase, phosphoglucose isomerase, invertase and glucokinase genes and the like in the glycolytic pathway, heat shock protein promoter, MFa-1 promoter, CUP 1 promoter, MET, the promoter of the TRP1 gene, the ADC1 gene (coding for the alcohol dehydrogenase I) or ADR2 gene (coding for the alcohol dehydrogenase II), acid phosphatase (PHO5) gene, isocytochrome c gene, a promoter of the yeast mating pheromone genes coding for the a- or α-factor, or the GAL/CYC1 hybrid promoter (intergenic region of the GAL1-GAL10 gene/Cytochromel gene) (Guarente et al. 1982). Promoters with transcriptional control that can be turned on or off by variation of the growth conditions include, e.g., PHO5, ADR2, and GAL/CYC1 promoters. The PHO5 promoter, for example, can be repressed or derepressed at will, solely by increasing or decreasing the concentration of inorganic phosphate in the medium. Some promoters, such as the ADH1 promoter, allow high-level constitutive expression of the gene of interest.

Any promoter capable of expressing in filamentous fungi may be used. Examples are a promoter induced strongly by starch or cellulose, e.g., a promoter for glucoamylase or a-amylase from the genus Aspergillus or cellulase (cellobiohydrase) from the genus Trichoderma, a promoter for enzymes in the glycolytic pathway, such as phosphoglycerate kinase (pgk) and glycerylaldehyde 3-phosphate dehydrogenase (gpd), etc.

Particular bacterial promoters include but are not limited to E. coli lac or trp, the phage lambda P_{L}, lacI, lacZ, T3, T7, gpt, and lambda P_{R} promoters.

Two principal methods for the control of expression are known, viz.: induction, which leads to overexpression, and repression, which leads to underexpression. Overexpression can be achieved by insertion of a strong promoter in a position that is operably linked to the target gene, or by insertion of one or more than one extra copy of the selected gene. For example, extra copies of the gene of interest may be positioned on an autonomously replicating plasmid, such as pYES2.0 (Invitrogen Corp., Carlsbad, Calif.), where overexpression is controlled by the GAL4 promoter after addition of galactose to the medium.

Several inducible promoters are known in the art. Many are described in a review by Gatz, Curr. Op. Biotech., 7:168 (1996) (see also Gatz, Ann. Rev. Plant. Physiol. Plant Mol. Biol., 48:89 (1997)). Examples include tetracycline repressor system, Lac repressor system, copper-inducible systems, salicylate-inducible systems (such as the PR1a system), glucocorticoid-inducible (Aoyama T. et al., 1997), alcohol-inducible systems, e.g., AOX promoters, and ecdysome-inducible systems. Also included are the benzene sulphonamide-inducible (U.S. Pat. No. 5364,780) and alcohol-inducible (WO 97/06269 and WO 97/06268) inducible systems and glutathione S-transferase promoters.

In addition to the use of a particular promoter, other types of elements can influence expression of transgenes. In particular, introns have demonstrated the potential for enhancing transgene expression.

Other elements include those that can be regulated by endogenous or exogenous agents, e.g., by zinc finger proteins, including naturally occurring zinc finger proteins or chimeric zinc finger proteins. See, e.g., U.S. Pat. No. 5,789,538, WO 99/48909; WO 99/45132; WO 98/53060; WO 98/53057; WO 98/53058; WO 00/23464; WO 95/19431; and WO 98/54311.

An enhancer is a DNA sequence that can stimulate promoter activity and may be an innate element of the promoter or a heterologous element inserted to enhance the level or tissue specificity of a particular promoter. An enhancer is capable of operating in both orientations (5= to 3= and 3= to 5= relative to the gene of interest coding sequences), and is capable of functioning even when moved either upstream or downstream from the promoter. Both enhancers and other upstream promoter elements bind sequence-specific DNA-binding proteins that mediate their effects.

Vectors for use in accordance with the present invention may be constructed to include an enhancer element. Constructs of the invention will also include the gene of interest along with a 3′ end DNA sequence that acts as a signal to terminate transcription and allow for the polyadenylation of the resultant mRNA.

As the DNA sequence between the transcription initiation site and the start of the coding sequence, i.e., the untranslated leader sequence, can influence gene expression, one may also wish to employ a particular leader sequence. Leader sequences are contemplated to include those that include sequences predicted to direct optimum expression of the attached gene, e.g., to include a consensus leader sequence that may increase or maintain mRNA stability and prevent inappropriate initiation of translation. The choice of such sequences will be known to those of skill in the art in light of the present disclosure.

In order to improve the ability to identify transformants, one may desire to employ a selectable or screenable marker gene as, or in addition to, the expressible gene of interest. “Marker genes” are genes that impart a distinct phenotype to cells expressing the marker gene and thus allow such transformed cells to be distinguished from cells that do not have the marker. Such genes may encode either a selectable or screenable marker, depending on whether the marker confers a trait that one can >select= for by chemical means, i.e., through the use of a selective agent (e.g., an antibiotic, or the like), or whether it is simply a trait that one can identify through observation or testing, i.e., by >screening=. Of course, many examples of suitable marker genes are known to the art and can be employed in the practice of the invention.

Included within the terms selectable or screenable marker genes are also genes that encode a “secretable marker” whose secretion can be detected as a means of identifying or selecting for transformed cells. Examples include markers that encode a secretable antigen that can be identified by antibody interaction, or even secretable enzymes that can be detected by their catalytic activity. Secretable proteins fall into a number of classes, including small, diffusible proteins detectable, e.g., by ELISA and small active enzymes detectable in extracellular solution.

Screenable markers that may be employed include, but are not limited to, a β-glucuronidase or uidA gene (GUS) that encode an enzyme for which various chromogenic substrates are known; a beta-lactamase gene (Sutcliffe, 1978), which encodes an enzyme for which various chromogenic substrates are known (e.g., PADAC, a chromogenic cephalosporin); a xylE gene (Zukowsky et al., 1983), which encodes a catechol dioxygenase that can convert chromogenic catechols; an alpha-amylase gene (Ikuta et al., 1990); a tyrosinase gene (Katz et al., 1983) that encodes an enzyme capable of oxidizing tyrosine to DOPA and dopaquinone that in turn condenses to form the easily detectable compound melanin; a beta-galactosidase gene, which encodes an enzyme for which there are chromogenic substrates; a luciferase (lux) gene (Ow et al., 1986), which allows for bioluminescence detection; or even an aequorin gene (Prasher et al., 1985), which may be employed in calcium-sensitive bioluminescence detection, or a green fluorescent protein gene (Niedz et al., 1995). Selectable nutritional markers may also be used, such as HIS3, URA3, TRP-1, LYS-2 and ADE2.

Any construct encoding a gene product that results in a recombinant cell useful in biocatalysis may be employed. Sources of genes for oxidoreductases include those from fungal cells belonging to the genera Aspergillus, Rhizopus, Trichoderma, Neurospora, Mucor, Penicillium, yeast belonging to the genera Kluyveromyces, Saccharomyces, Schizosaccharomyces, Trichosporon, Schwanniomyces, bacteria, plants, vertebrates and the like. In one embodiment, the construct is on a plasmid suitable for extrachromosomal replication and maintenance. In another embodiment, two constructs each encoding an enzyme, e.g., for a “coupled” reaction, are concurrently or sequentially introduced to a cell so as to result in stable integration of the constructs into the genome.

In one embodiment, the microbial cells of the invention express xylose reductase, or xylose reductase and a dehydrogenase, e.g., glucose, alcohol or formate dehydrogenase, such as one that requires NAD(P) or NAD(P)H.

In one embodiment, microbial cells such as yeast cells, e.g., Pichia cells, are spray-dried and employed for production of xylitol. In one embodiment, the microbial cells express recombinant xylose reductase. Xylose reductases within the scope of the invention include but are not limited to those from fungi, plants, e.g., dicots or monocots, or yeast, e.g., including but not limited to those from Ascomcota including Pezizomycotina, Arthoniomycetes, Dothideomycetes, Eurotiomycetes, Laboulbeniomycetes, Lecanoromycetes, Leotiomycetes, Lichinomycetes, Orbiliomycetes, Pezizomycetes, Sordariomycetes, Lahmiales, Medeolariales, Triblidiales, Geoglossaceae, Saccharomycotina, Saccharomycetes, Taphrinomycotina, Neolectomycetes, Pneumocystidomycetes, Schizosaccharomycetes, and Taphrinomycetes, as well as Basidiomycota such as Pucciniomycotina, Ustilaginomycotina, Agaricomycotina, Incertae sedis, Wallemiomycetes and Entorrhizomycetes;

gram positive bacteria, e.g., Actinobacteria, Firmicutes and Tenericutes; and gram negative bacteria, e.g., Aquificae, Bacteroidetes/Chlorobi, Chlamydiae/Verrucomicrobia, Deinococcus-Thermus, Fusobacteria, Gemmatimonadetes, Nitrospirae, Proteobacteria, Spriochaetes, Synergistetes, Acidobacteria, Chloroflexi, Chrysiogenetes, Cyanobacteria, Deferribacteres, Dictyoglomi, Fibrobacteres, Planctomycetes, Thermodesulfobacteria, and Thermotogae, as well as aquatic organisms such as fish, e.g., Salmo salar.

In one embodiment, the microbial cells, e.g., yeast cells, express a recombinant dehydrogenase such as glucose or formate dehydrogenase, optionally in addition to recombinant xylose reductase. Dehydrogenases within the scope of the invention include but are not limited to those from Enterobacter asburiae, Aspergillus terreus, Bacillus megaterium, Bacillus subtilis, Burkholderia cepacia, Enterobacter asburiae, Haloferax mediterranei, Sulfolobus solfataricus, Sulfolobus tokodaii, Bacillus licheniformis, Picrophilus torridus, and Gluconacetobacter diazotrophicus, for example, those from Staphylococcus, Streptococcus, Enterococcus, Bacillus, Corynebacterium, Nocardia, Clostridium, Actinobacteria, Listeria, Mycoplasma, Escherichia coli, Salmonella, Shigella, Enterobacteriaceae, Pseudomonas, Moraxella, Helicobacter, Stenotrophomonas, Bdellovibrio, acetic acid bacteria, Legionella, Wobachia, cyanobacteria, spirochaetes, green sulfur and green non-sulfur bacteria, e.g., Neisseria Spp., Moraxella catarrhalis, Hemophilus influenza, Klebsiella pneumoniae, Legionella pneumophila, Pseudomonas aeruginosa, Proteus mirabilis, Enterobacter cloacae, Serratia marcescens, Helicobacter pylori, Salmonella enteritidis, Salmonella typhi, or Acinetobacter baumannii, or plants.

In one embodiment, a spray-dried preparation of Pichia suitable for biocatalysis is provided. In another embodiment, a spray-dried preparation of Saccharomyces suitable for biocatalysis is provided. It is envisioned that spray-dried preparations of yeast other than Pichia or Saccharomyces may be employed for biocatalysis. In one embodiment, the yeast comprises at least one recombinant oxidoreductase. For example, the recombinant enzyme may be a heterologous xylose reductase. In one embodiment, the yeast comprises a heterologous xylose reductase and a native (endogenous) dehydrogenase such as native glucose dehydrogenase. In one embodiment, the yeast comprises a heterologous xylose reductase and a heterologous dehydrogenase such as a heterologous glucose dehydrogenase. In one embodiment, the yeast does not express a recombinant enzyme, e.g., wild-type (or otherwise nonrecombinant) yeast such as Saccharomyces may be employed for biocatalysis.

In one embodiment, a spray-dried preparation of prokaryotic cells such as bacteria including E. coli and Pseudomonas cells for biocatalysis is provided. For example, a recombinant E. coli strain comprises at least one recombinant oxidoreductase. In one embodiment, the E. coli strain is transformed with a prokaryotic vector derived from pET32.

To prepare recombinant strains of microbes, the microbial genome is augmented or a portion of the genome is replaced with an expression cassette. For biocatalysis, the expression cassette comprises a promoter operably linked to an open reading frame for at least one enzyme that mediates the biocatalysis. For example, the expression cassette may encode a heterologous xylose reductase. In one embodiment, the microbial genome is transformed with at least two expression cassettes, e.g., one expression cassette encodes a heterologous xylose reductase and another encodes a heterologous dehydrogenase such as a heterologous glucose or formate dehydrogenase. The expression cassettes may be introduced on the same or separate plasmids or the same or different vectors for stable integrative transformation.

Recombinant or native (nonrecombinant) microbes expressing one or more enzymes that mediate a particular enzymatic reaction are expanded to provide a microbial cell suspension. In one embodiment, the suspension may be separated into a liquid fraction and a solid fraction which contains the cells, e.g., by centrifugation or use of a membrane, prior to spray drying. The microbial cell suspension is spray-dried under conditions effective to yield a spray-dried microbial cell preparation suitable for biocatalysis. In one embodiment, the spray drying includes heating an amount of the cell suspension flowing through an aperature. The conditions described in the examples below were set based on small-scale instrument capacity, e.g., low evaporation capacity (e.g., 1.5 Kg water/hour). Thus, the ranges below are exemplary only and may be different at a manufacturing scale where evaporation capacity may reach over 1000 Kg water/hour. For example, for a small scale instrument, the feed (flow) rate may be about 1 mL/minute to about 30 mL/minute, e.g., about 2 mL/minute up to about 20 mL/minute. Flow rates for large scale processes may be up to or greater than 1 L/minute. In one embodiment, the suspension is dried at about 50° C. up to about 225° C., e.g., about 100° C. up to about 200° C. The cell suspension prior to spray drying may be at about 5 mg/L up to about 800 mg/L, for instance, about 20 mg/L up to about 700 mg/L, or up to or greater than 2000 mg/L. The upper limit of the concentration of cells employed is determined by the viscosity of the cells and the instrument. The microbial cell suspension may be an E. coli cell suspension, a Pseudomonas cell suspension, a Pichia cell suspension or a Saccharomyces cell suspension. In one embodiment, air flow is about 500 L/hr to about 800 L/hr, e.g., about 700 L/hr. The process to prepare a spray-dried microbial cell preparation may include any flow rate, any temperature and any cell concentration described herein, as well as other flow rates, temperatures and cell concentrations.

Once desirable native or recombinant microbial cells are spray-dried, they may be stored for any period of time under conditions that do not substantially impact the activity or cellular location of enzymes to be employed in biocatalysis. Storage periods include hours, days, weeks, and up to at least 2 months.

In one embodiment, the yeast cells to be spray-dried and used in biocatalysis are Pichia cells having a heterologous xylose reductase DNA expressed from an AOX1 promoter (single recombinant), for instance, Pichia pastoris cells having P. stipitis xylose reductase DNA, and optionally a heterologous dehydrogenase DNA, e.g., Bacillus glucose dehydrogenase DNA, where each enzyme is expressed from AOX1 and/or AOX2 promoters, or other inducible promoters. Those cells exhibit prokaryotic growth rates, produce the heterologous enzymes intracellularly, have high production rates (grams per liter), have controllable expression (induced by MeOH/repressed by glycerol), and their fermentation is readily scaled up to 100 to 10,000 L.

The invention will be further described by the following non-limiting examples.

### EXAMPLE 1

### Spray-Drying

Equipment

A Buchi B-190 spray-dryer with a pneumatic nozzle cleaner may be used for all spray-drying procedures. Controlled operating parameters may include: feed concentration, feed rate, air flow, temperature, and vacuum. The air flow may be set to 700 L/hour and the vacuum may be constant at −50 mbar. Experimental feed concentrations may range from 30 to 600 mg/mL, feed rates may span from 5 to 15 mL/minute, and operating temperatures may be from 120 to 195° C.

Procedure

Pichia pastoris cells are washed, centrifuged, and stored at −80° C. These cells may be obtained from the freezer, allowed to thaw at room temperature, and excess water removed by blotting the cells on filter paper. The blotted cells are weighed into several containers to yield desired cell concentrations after dilution to a fixed volume. The blotted cells are diluted with deionized water. The containers are agitated, forming cell suspensions at specified concentrations to be used as feeds to the spray-dryer.

Air flow is initiated to the spray-dryer at 700 L/hour, the aspirator is set to −50 mbar, and the heater is powered on. Deionized water is pumped through the spray nozzle in place of the cell suspension. The feed rate is set to the desired experimental value, and the heater is adjusted to yield the correct operating temperature. After reaching temperature equilibrium, the deionized water feed is replaced by the specified cell suspension. The spray-drying process may last between 6 and 20 minutes based on the feed rate used. When the 100 mL cell suspension has been processed, the feed pump and heater are stopped, and air flow is allowed to cool the machine to 70° C. Then, the aspirator and air supply are shut down. The collection vessel is removed, and the Pichia cells are transferred to a scintillation vial. These vials are stored at ambient temperature in a desiccator. The feed line is flushed with deionized water and the glassware rinsed before spray-drying at other conditions.

### Experiment

Temperature shifts associated with the spray-drying process may break-open the Pichia cell membranes and rapid moisture loss may destabilize the recombinant enzymes. Feed concentration, feed rate, and operating temperature may be varied for their relative effects on enzyme activity.

A full-factorial experiment may be designed with three factors (feed concentration, feed rate, and operating temperature). A high and low value is investigated for each of the three factors. This results in a total of eight experimental runs shown in Table 1.

TABLE 1

| Temperature | Feed Rate | Feed Concentration |
|---|---|---|
| [C.] | [mL/min] | [mg/mL] |
| 120 | 5 | 30 |
| 120 | 5 | 100 |
| 120 | 15 | 30 |
| 120 | 15 | 100 |
| 150 | 5 | 30 |
| 150 | 5 | 100 |
| 150 | 15 | 30 |
| 150 | 15 | 100 |

Enzyme activities are the two responses used to screen these spray-dryer operating parameters.

JMP statistical software (SAS Company) may be used to plan the screening experiment and analyze data. A constructed model incorporates feed concentration, feed rate, temperature, and all three interaction parameters (feed concentration*feed rate, feed concentration*temperature, and feed rate*temperature). Standard least squares analysis may be used to minimize residual error. Three absorbance measurements for each experimental condition (two replicates) are included in the statistical analysis. Leverage plots gauge relative influence of the spray-drying parameters on enzyme activity.

For instance, increasing temperature over, e.g., 150° C., may result in a decrease in activity, while elevating feed rate or feed concentration may raise activity. Moreover, lower temperatures, e.g., spray-drying at 120° C., may produce a “caked” powder (due to residual moisture) when operating at higher feed rates. As an alternative, higher feed concentrations may result in the retention of enzyme activity when spray-drying above 120° C.

### Experiment

A full factorial experiment may also be designed with two factors (e.g., three different temperatures and three different feed concentrations) (Table 2).

TABLE 2

| Temperature | Feed Concentration |
|---|---|
| [C.] | [mg/mL] |
| 120 | 100 |
| 120 | 200 |
| 120 | 400 |
| 150 | 100 |
| 150 | 200 |
| 150 | 400 |
| 195 | 100 |
| 195 | 200 |
| 195 | 400 |

JMP statistical software is used to develop a model based on temperature, feed concentration, and one interaction parameter (temperature*feed concentration). Higher feed concentrations and higher operating temperatures are tested. The maximum controlled temperature (at the specified feed rate) may be 195° C. This upper temperature limit was studied to enhance results from increasing the feed concentration and to determine a temperature optimum. Six absorbance measurements for each experimental condition (5 replicates) are included in the statistical analysis. Leverage plots are prepared for enzyme activity vs. the spray-drying parameters.

The benefit of spray-drying at higher feed concentrations may not be evident by observing the feed concentration leverage plot. However, there are two benefits to increasing the feed concentration: a) more P. pastoris cells can be processed per unit volume, and b) the temperature*feed concentration interaction plot may show higher feed concentrations helped achieve comparable activity for the 120 and 150° C. runs. Spray-drying at 150° C. may yield a more uniform biocatalyst powder with less residual moisture than operating at 120° C.

### Experiment

The best feed rate (e.g., 15 mL/minute) and best temperature (e.g., 150° C.) from previous studies may be used. Increasing feed concentration can be beneficial by reducing processing volume and helping maintain enzyme activity if spray-drying at temperatures above 150° C. was used. Feed concentrations of 200 400 and 600 mg/mL are investigated. The viscosity of the higher concentration may slow the feed rate.

Enzyme stability studies, cell permeabilization studies, enzyme leaching studies may be investigated. For instance, the spray-dried cells are stored at room temperature in a desiccator (to prevent moisture contamination). These cells may be assayed for enzyme activity over a period of time to observe possible activity loss and quantify enzyme stability. In addition, spray-dried cells may be compared to several other methods of cell preparation which permeabilize cells, for instance, BAC treated cells. In the case of xylitol reductase in non-spray dried cells, BAC inhibited the reaction (PG 4000-4 cells, described below, at 60 mg/mL in the presence of BAC (0.1%) lost activity after 4 hours, provided for a slow eaction rate, e.g., 80 mM xylitol in 20 hours and required NAD+ addition). Further enzyme leaching studies determine if enzymes in coupled reactions contained within the whole cell biocatalyst are protected from shear forces in the reactor and are spatially close so they can work together.

### EXAMPLE 2

Current issues with microbial xylitol production are providing a consistent feed stream, inhibiting fermentation, poor cell yield, long fermentation times and low yield and recovery. Moreover, the separation of xylitol from other metabolites is problematic. Separating fermentation from biocatalysis has a number of advantages which are not specific to xylitol production, e.g., Pichia high cell density fermentation has been optimized in the absence of antibiotics, spray-dried Pichia cells are porous but do not leach enzymes, enzymes are stable and the cells may be employed more than once. Thus, a dried powder may be used for xylitol production with recycling of the catalyst and optionally with addition at one or more cycles of NAD(P)⁺, glucose or formate. For efficient conversions, e.g., conversions of at least 20% or more, e.g., at least 30%, 40% 50%, 60%, 70% or 80%, the product stream is relatively clean, e.g., needs minimal purification.

The xylose reductase (XR) employed in the spray-dried cells may be native or recombinant. Exemplary recombinant XRs include those from Neurospora crassa (NADPH-specific), C. intermedia, C. parapsilosis (e.g., from a strain that is NADH-specific), C. tropicalis, C. tenuis, P. tannophilus, P. stipitis (can employ NADH or NADPH) and S. cerevisiae. To enhance expression of XR, the recombinant XR gene may be under the control of an inducble promoter, e.g., a methanol inducible promoter. Pichia cells expressing XR are grown on glycerol/methanol and then spray-dried. The spray-dried preparation is combined with a hemicellulose hydroslyate optionally in the present of NAD(P)⁺.

The microbial cells may also express a NADH/NADPH regenerating enzyme, e.g., a dehydrogenase (DH). The DH employed in the spray-dried cells may be native or recombinant. Exemplary recombinant DHs include formate DH (FDH), e.g., Candida boidinii FDH, glucose-6-phosphate DH, phosphate DH, glucose DH (GDH), e.g., B. megaterium GDH, and aldehyde DH. In one embodiment, P. stipitis XR is employed with native P. pastoris FDH or Bacillus subtilis GDH. In one embodiment, C. parapsilosis XR is employed with native P. pastoris FDH or Bacillus subtilis GDH. In one embodiment, N. crassa XR is employed with P. pastoris FDH or Bacillus subtilis GDH. Regardless of the source of the XR or DH (native or recombinant), both are stabilized in spray-dried cells. Thus, both coupled and sequential reactions for biocatalysis of any product may be conducted in spray-dried cells.

### Materials and Methods

Materials. All chemical reagents were purchased from Sigma-Aldrich (St. Louis, Mo.) and Fisher Scientific (Pittsburgh, Pa.) unless otherwise noted. PCR primers were purchased from Integrated DNA Technologies (Coralville, Iowa). Turbo Pfu DNA polymerase and corresponding buffer (Stratagene, La Jolla, Calif.) were used in all PCR reactions unless otherwise noted. Other molecular biology reagents were purchased from Invitrogen (Carlsbad, Calif.), New England Biolabs (Ipswich, Mass.), Fermentas (Glen Burnie, Md.), Promega (Madison, Wis.), Qiagen (Valencia, Calif.), and Epicentre (Madison, Wis.).

Pichia pastoris expression plasmid. P. pastoris expression plasmid pPIC3.5K was purchased from Invitrogen. This expression plasmid needed to be modified before cloning of xylose reductase (XR) gene and glucose dehydrogenase gene (gdh). First, an AfeI (also known as Eco47III) restriction enzyme site at nucleotide position 1524 of pPIC3.5K was removed by site-directed mutagenesis using primers pPIC35sdm-F (5′-GTCACTATGGCGTGCTGCTGGATCCATATG CGTTGATGCAATTTC-3′; SEQ ID NO:1) and pPIC35 sdm-R (5′-GAAATTGCATCAACG CATATGGATCCAGCAGCACGCCATAGTGAC-3′; SEQ ID NO:2). The 50 μL, site-directed mutagenesis reaction mixture contained 5 μL, 10× Turbo Pfu DNA polymerase buffer, 5 ng pPIC3.5K, 168 nM primer pPIC35sdm-F, 168 nM primer pPIC35sdm-R, 200 μM dNTP, and 2.5 U Turbo Pfu DNA polymerase. The reaction was incubated in a PCR thermocycler under the following conditions: (1) 1 cycle at 95° C. for 30 seconds; (2) 18 cycles at 95° C. for 30 seconds→55° C. for 1 minute→68° C. for 9 minutes; (3) 1 cycle at 4° C. for 2 minutes. After that, 20 U of DpnI was added to the reaction mixture and incubated at 37° C. for 1 hour. DpnI removed all pPIC3.5K template molecules in the reaction but left the plasmid molecules generated in the site-directed mutagenesis reaction intact. An aliquot of the reaction (2.5 μL) was then transformed into E. coli JM109 cells by electroporation for the recovery of the site-directed mutated plasmid pPIC3.5Kx. Successful removal of the AfeI site was confirmed by comparing the AfeI digestion pattern of pPIC3.5K and pPIC3.5Kx. Plasmid pPIC3.5K has 2 AfeI sites and digestion by AfeI yielded 2 DNA fragments of 4,168 and 4,836 bp. On the contrary, pPIC3.5Kx has only a single AfeI site and so it was linearized into a single 9,004 by DNA fragment after AfeI digestion. Successful removal of the AfeI site was further confirmed by comparing the BamHI digestion pattern of pPIC3.5K and pPIC3.5Kx. Plasmid pPIC3.5K has only 1 BamHI site and thus BamHI digestion produced a single 9004 by DNA fragment. A BamHI site was engineered into primers pPIC35sdm-F and -R (underlined sequence) and thus pPIC3.5Kx would contain 2 BamHI sites. BamHI digestion of pPIC3.5Kx resulted in 2 DNA fragments of 586 and 8,418 bp.

After confirming the identity of pPIC3.5Kx, it was sequentially digested by restriction enzymes AfeI and BstZ171. The double digestion yielded 2 blunt-end DNA fragments (7,911 and 1,093 bp). The 7,911 by DNA fragment was purified from agarose gel by Qiagen gel extraction kit. The purified fragment was then self-ligated by using T4 DNA ligase (New England Biolabs) and transformed into E. coli JM109 by electroporation. The resultant 7,911-bp plasmid is designated as pPIC4Kx and it was used in all subsequent cloning experiment.

Cloning of the Bacillus subtilis glucose dehydrogenase gene (gdh). Plasmid pUC19-gdh was a generous gift from Dr. Jack Rosazza (Univ. of Iowa). The gdh gene sequence contained an internal AsuII (also known as BstBI) restriction site which interfered with cloning. Therefore, site-directed mutagenesis was used to remove this internal AsuII from gdh (without changing protein sequence) with primers gdh-sdm-F (5′-GCCTGGCTTGCTTCCAAGGAAGCCAGCTA-3′; SEQ ID NO:3) and gdh-sdm-R (5′-TAGCTGGCTTCCTTGGAAGCAAGCCAGGC-3′; SEQ ID NO:4). The 50-μL site-directed mutagenesis reaction mixture contained 5 μL 10× Turbo Pfu DNA polymerase buffer, 10 ng pUC19-gdh, 262 nM primer gdh-sdm-F, 262 nM primer gdh-sdm-R, 200 μM dNTP, and 2.5 U Turbo Pfu DNA polymerase. The reaction was incubated in a PCR thermocycler under the following conditions: (1) 1 cycle at 95° C. for 30 seconds; (2) 16 cycles at 95° C. for 30 seconds→55° C. for 1 minute→68° C. for 4 minutes; (3) 1 cycle at 4° C. for 2 minutes. After that, 20 U of DpnI was added to the reaction mixture and incubated at 37° C. for 1 hour. DpnI removed all pUC19-gdh template molecules in the reaction but left the plasmid molecules generated in the site-directed mutagenesis reaction intact. An aliquot of the reaction (2.5 μL) was then transformed into E. coli JM109 cells by electroporation for the recovery of the site-directed mutated plasmid, designated as pUC19-gdh-sdm. Successful removal of the AsuII site within gdh coding sequence was confirmed by DNA sequencing.

After that, the gdh-sdm gene was amplified from pUC19-gdh-sdm by PCR using primers gdh-F (5′-CGCGCGTTCGAACAAAATGTACCCGGATTTAA AAGG-3′; SEQ ID NO:5) and gdh-R (5′-GAATTAGAATTCTTAACCGCGGCCTGCCTGGA-3′; SEQ ID NO:6). The PCR thermal profile was (1) 1 cycle of 3 minutes at 95° C.; (2) 30 cycles of 30 seconds at 95° C., 30 seconds at 58° C., and 60 seconds at 72° C.; (3) 1 cycle of 10 minutes at 72° C. The PCR product was gel-purified, followed by AsuII and EcoRI digestion (restriction sites engineered in gdh-F and gdh-R primers and were underlined). The restriction digested PCR product was ligated into plasmid pPIC4Kx pre-digested with AsuII and EcoRI, forming plasmid pPIC4Kx-gdh-sdm. DNA sequencing confirmed successful cloning of the gdh-sdm gene into pPIC4Kx, with no mutation introduced in gdh-sdm coding sequence due to cloning procedures.

Cloning of the Pichia stipitis xylose reductase gene (PsXR). Genomic DNA of Pichia stipitis was purchased from American Type Culture Collection (Manassas, Va.) and it was used as template for PCR amplification of the PsXR gene, with primers PsXR-F (5′-GCGCGCTTCGAACAAAATGCCTTCTATT AAGTTGAA-3′; SEQ ID NO:7) and PsXR-R (5′-GGCGAGCAATTGTTAGACGAAGATAGGA ATCT-3′; SEQ ID NO:8). The PCR thermal profile was (1) 1 cycle of 3 minutes at 95° C.; (2) 30 cycles of 30 seconds at 95° C., 30 seconds at 58° C., and 60 seconds at 72° C.; (3) 1 cycle of 10 minutes at 72° C. The PCR product was gel-purified, followed by AsuII and MfeI digestion (restriction sites engineered in PsXR-F and PsXR-R primers and are underlined). The restriction digested PCR product was ligated into plasmid pPIC4Kx pre-digested with AsuII and EcoRI (EcoRI cut site is compatible with MfeI cut site), forming plasmid pPIC4Kx-PsXR. DNA sequencing confirmed successful cloning of the PsXR gene into pPIC4Kx with no mutation introduced in PsXR coding sequence due to cloning procedures.

Cloning of the Candida parapsilosis xylose reductase gene (CpXR). C. parapsilosis KFCC-18075 is a “mutant” of C. parapsilosis ATCC 22019, but the nature of this “mutation” is unclear (Oh et al., 1998). The CpXR isolated from C. parapsilosis KFCC-18075 was reported to be a NADH-specific xylose reductase. Since strain KFCC-18075 was unavailable, strain ATCC 22019 was purchased. Strain ATCC 22019 was cultivated in YM broth (Becton, Dickinson and Company, Sparks, Md.) and genomic DNA was extracted from the culture using Puregene yeast genomic DNA purification kit (Qiagen). A pair of PCR primers CpXR-F and -R were designed based on C. parapsilosis KFCC-18075 CpXR gene sequence (Genbank accession No. AY193716). However, we failed to amplify any PCR product after numerous attempts. It is possible that the CpXR gene in strain KFCC-18075 is significantly different from that of strain ATCC 22019. Meanwhile, by searching the on-going genome sequencing project of Candida parapsilosis isolate 317 (http://www.sanger.ac.uk/sequencing/Candida/parapsilosis/), an uncharacterized XR gene was identified. Therefore, a new pair of degenerate PCR primers CpXR-F2 (5′-ATGTCNATYAARTTRAAYTCNGG-3′; SEQ ID NO:9) and CpxR-R2 (5′-CTARACAAARAYTGGAATGT-3′; SEQ ID NO:10) was designed to amplify a XR gene from genomic DNA prepared from ATCC 22019. This PCR reaction was set up using FailSafe PCR buffer G (Epicentre) in combination with Taq DNA polymerase (New England Biolabs). The PCR thermal profile was (1) 1 cycle of 3 minutes at 95° C.; (2) 30 cycles of 30 seconds at 95° C., 30 seconds at 40° C., and 60 seconds at 72° C.; (3) 1 cycle of 10 minutes at 72° C. The 1 kb PCR product was directly cloned into PCR product cloning vector pGEM-Teasy (Promega). DNA sequencing confirmed the resultant plasmid, pGEM-Teasy+CpxR F2/R2 B6, contained the XR gene identified in C. parapsilosis genome sequencing project.

PCR primers CpXR-F3 (5′-CGCGGCTTCGAACAAAATGTCGAT TAAATTAAATTC-3′; SEQ ID NO:11) and CpXR-R3 (5′-TAAGCTGAA TTCCTAGACAAAGATTGGAATGTGATC-3′; SEQ ID NO:12) were used to amplify the CpXR gene from pGEM-Teasy+CpxR F2/R2 B6 using FailSafe PCR buffer G and Taq DNA polymerase. The PCR thermal profile was (1) 1 cycle of 3 minutes at 95° C.; (2) 30 cycles of 30 seconds at 95° C., 30 seconds at 55° C., and 60 seconds at 72° C.; (3) 1 cycle of 10 minutes at 72° C. The PCR product was gel-purified, followed by AsuII and EcoRI digestion (restriction sites engineered in CpXR-F3 and CpXR-R3 primers and were underlined). The restriction digested PCR product was ligated into plasmid pPIC4Kx pre-digested with AsuII and EcoRI, forming plasmid pPIC4Kx-CpXR. DNA sequencing confirmed successful cloning of the CpXR gene into pPIC4Kx, with no mutation introduced in CpXR coding sequence due to cloning procedures.

Cloning of the Neurospora crassa xylose reductase gene (NcXR). The NcXR gene is (Genbank accession No. NW_{—}001849801.1) composed of 3 exons which are 142, 791, and 486 by in size. Coding sequences of NcXR are located in exon 1, exon 2, and the first 36 nucleotides of exon 3. So, the complete NcXR ORF is 969 nucleotides in length.

The crossover PCR technique described by Link et al. (1997) was used to “paste” exons 1 and 2 together. A N. crassa cosmid clone G 1-F 11 that contained the NcXR gene was purchased from the Fungal Genetics Stock Center at the University of Missouri, Kansas City. A forward primer (Ex1 out: 5′-ACGCAGTGAGGGGACAACATGAGCCGAAGT-3′; SEQ ID NO:12) was designed in region 5′ to the ATG start codon of NcXR in exon 1. In combination with reverse prime Ex1in (5′-CTCAACCTCGTTGCCGTAGTCGCAGGCAC CATCGAAGAGG-3′; SEQ ID NO:13), a PCR product that contained exon 1 DNA sequence was amplified from cosmid G1-F11 according to the crossover PCR procedure (Link et al., 1997), using Turbo Pfu DNA polymerase and the following PCR thermal profile: 30 cycles of 30 seconds at 95° C., 30 seconds at 58° C., and 60 seconds at 72° C. Another pair of primers Ex2in(5′-CCTCTTCGATGGTGCCTGCGACTACGGCAACGAGGTTGAG-3′; SEQ ID NO:14) and Ex2out (5′-GTTGGTGGGCTGGTTGAAGCGGATGCC-3′; SEQ ID NO: 15) was used to amplify exon 2 from cosmid G1-F11 by the same crossover PCR procedure. These 2 PCR products (0.1 μL of each) were mixed together with primers Ex1out and Ex2out for amplification of a exon 1 and 2 in-frame fusion product by the following PCR thermal profile: (1) 1 cycle of 3 minutes at 95° C.; (2) 5 cycles of 30 seconds at 95° C., 30 seconds at 55° C., and 90 seconds at 72° C.; (3) 25 cycles of 30 seconds at 95° C., 30 seconds at 58° C., and 90 seconds at 72° C.; (4) 1 cycle of 10 minutes at 72° C. This fusion product was named PCR #F.

PCR #F was gel purified by a Qiagen gel extraction kit. The purified product was used as template for a regular PCR using primers NcXR-F (5′-GCGCGCTTCGAACAAAATGGTTCCTGCTATCAAGCT-3′; SEQ ID NO:16) and Ex1&2in (5′-AGGTTCTCAGCGGAGAAGTAGTTGGTGG GCTGGTTGAAGCG-3′; SEQ ID NO:17) with Turbo Pfu DNA polymerase. This 0.9-kb PCR product was gel purified and used as template in a final round of PCR with primers NcXR-F and Ex123-R (5′-CTAACCGAAAATCC AGAGGTTCTCAGCGGAGAAGTA-3′; SEQ ID NO:18). This PCR reaction mixture contained FailSafe buffer E (Epicentre), 2.5 U Taq DNA polymerase, 2.5 U Turbo Pfu DNA polymerase, and 300 nM of each primer. The PCR product was then gel-purified and concentrated into a final volume of 4 μL, and mixed with 0.6 μL 10× Taq DNA polymerase buffer, 0.5 μL 3 mM dATP, and 0.6 μL Taq DNA polymerase. This mixture was incubated at 70° C. for 30 minutes. After that, the whole reaction mixture was used for ligation with 2 μL of pGEM-Teasy vector (Promega) at 16° C. for 16 hours. The ligation reaction was then transformed into chemical competent E. coli TOPO10 cells. Plasmid DNA was recovered from these transformant and one of them, pGEM-Teasy NcXR-F/Ex123-R W26, contained the complete ORF for NcXR. DNA sequencing confirmed no mutation was present in the NcXR coding sequence.

The NcXR ORF was released from pGEM-Teasy NcXR-F/Ex123-R W26 using AsuII and EcoRI (AsuII site engineered on primer NcXR-F; EcoRI site located on pGEM-Teasy plasmid, 10 nucleotides 3′ to the stop codon of NcXR). This AsuII-EcoRI fragment was ligated into plasmid pPIC4Kx pre-digested with AsuII and EcoRI, forming plasmid pPIC4Kx-NcXR. DNA sequencing confirmed no mutation was introduced into NcXR coding sequence by the cloning procedures.

Generation of expression plasmid with both XR and GDH expression cassettes. In all the P. pastoris expression plasmids, the gene of interest (either XR or gdh) is flanked by an AOX1 promoter (P_{AOX1}) and the AOX1 transcription terminator (AOX_{TT}). Each P_{AOX1}-gene-AOX_{TT} unit is referred as an expression cassette. The gdh expression cassette was released from pPIC4Kx-gdh-sdm by BamHI and Bg/II digestion. This cassette was cloned into pPIC4Kx-PsXR and pPIC4Kx-CpXR at the BamHI site (Bg/II and BamHI are compatible restriction enzymes), forming plasmids pPIC4Kx-PsXR-gdh and pPIC4Kx-CpXR-gdh, respectively. The gdh expression cassette was cloned into the Bg/II site of pPIC4Kx-NcXR, producing pPIC4Kx-gdh-NcXR.

Electroporation of P. pastoris GS115. Plasmids pPIC4Kx-PsXR-gdh, pPIC4Kx-CpXR-gdh, pPIC4Kx-gdh-NcXR, pPIC4Kx-PsXR, pPIC4Kx-CpXR, pPIC4Kx-NcXR, and pPIC4Kx were linearized by BspEI before electroporation. The linearized plasmids were individually transformed into electrocompetent P. pastoris GS115 (a His⁻ strain) prepared according to the procedure reported by Wu and Letchworth (2004). The transformed cells were then plated on minimal dextrose-sorbitol agar plates (1.34% yeast nitrogen base with ammonium sulfate but without amino acids, 4×10⁻⁵% biotin, 2% dextrose, 1 M sorbitol, and 2% agar) and incubated at 30° C. for 5-7 days. Expression plasmids integrated into GS115 genome would render a His⁺ phenotype to the transformants and allowed the transformants to grow on minimal dextrose-sorbitol agar without histidine supplementation.

Screening for transformants with multiple copies of expression plasmids. Multiple plasmid integration events occur spontaneously in P. pastoris and high copy number of plasmid integrations often correlates to higher levels of protein expression in P. pastoris. His⁺ transformants that grew on minimal dextrose-sorbitol agar were pooled together and plated on YPD agar (1% yeast extract, 2% peptone, 2% dextroxse, and 2% agar) containing geneticin by the following procedure:

1. Pipette 1 to 2 ml sterile water over the His⁺ transformants (from each expression plasmid electroporation) on each minimal dextrose-sorbitol plate.

2. Resuspend the His⁺ transformants into the water by using a sterile spreader and running it across the top of the agar.

3. Transfer and pool the cell suspension into a sterile, 50 ml conical centrifuge tube and vortex briefly.

4. Determine cell density of the cell suspension using a spectrophotometer (1 OD₆₀₀ unit about 5×10⁷ cells/ml).

5. Plate 10⁵ cells on YPD plates containing geneticin at a final concentration of 0.25, 0.5, 1.0, 1.5, 2.0, 3.0, and 4.0 mg/ml.

6. Incubate plates at 30° C. and check daily. Geneticin-resistant colonies will take 2 to 5 days to appear.

Colonies that grew on YPD-geneticin plates were streaked for purity on minimal dextrose-sorbitol agar plates. After obtaining single colonies, they were transferred back to YPD-geneticin agar to ensure the isolated colonies were resistant to geneticin. Isolated geneticin-resistant strains were chosen for protein expression study.

Expression study of selected transformants. Five transformants generated from each expression plasmid were chosen for protein expression study. Among the five transformants, two were resistant to 1 mg/mL of geneticin and three were resistant to 4 mg/mL geneticin. A single colony of each transformant was used to inoculate 20 mL BMGY broth (1% yeast extract, 2% peptone, 100 mM potassium phosphate (KPi) (pH 6), 1.34% yeast nitrogen base with ammonium sulfate but without amino acids, 4×10⁻⁵% biotin, and 1% glycerol). The cultures were incubated at 30° C. for 16 hours with orbital shaking at 300 rpm. In the next day, the BMGY cultures were used to inoculate 40 mL BMMY broth (same as BMGY except 0.5% methanol replaced 1% glycerol) in 500 mL baffled flasks. Methanol in the BMMY broth served as carbon/energy source for the cells as well as the inducer for protein expression. The BMMY cultures were incubated at 30° C. for 48 hours with orbital shaking at 300 rpm. After 24 hours, methanol was added to the BMMY cultures to a final concentration of 0.5% to maintain induction. At 24 and 48 hours, 1 mL of cells were sampled from each culture for measuring the cell density and protein expression levels. At 48 hours, all the cells in each culture were harvested by centrifugation at 4,000×g for 5 minutes. The cell pellets were stored at −80° C. until they were lysed for enzyme activity assays.

The 1 mL cell samples collected at 24 and 48 hours post induction were resuspended in 196 μL of Y-PerR Plus yeast protein extraction reagent (Thermo Scientific), 2 μL 0.5 M EDTA, and 2 μL 100× Halt Protease inhibitor cocktail (Thermo Scientific). To each cell pellet, an equal volume of glass beads (0.5 mm) was also added. The glass beads-cells suspensions were then vortexed vigorously for 30 seconds and then immediately chilled on ice for 30 seconds. This vortex-chilling procedure was repeated 7 more times. After that, the whole suspensions were incubated at 45° C. for 15 minutes with shaking at 300 rpm. Finally, the suspensions were centrifuged at 13,000 rpm for 2 minutes. The supernatants were saved as cell extracts. Protein concentrations in cell extracts were determined by Bradford assay (Bio-Rad) with bovine serum albumin as standard. Fifteen μg of protein from each cell extracts were loaded onto 10% SDS-PAGE gels (Bio-Rad) for analyses of XR and GDH expressions. Protein bands on gels were visualized after staining with GelCode Blue staining reagent (Thermo Scientific).

To generate larger quantities of cell extracts for enzyme activity assays, the major cell pellets from BMMY cultures were suspended in 7 mL 50 mM KPi buffer (pH 6.0). Half of the cell suspension was saved at −80° C. To the second half of cell suspension, 35 μL 100× Halt Protease inhibitor cocktail (Thermo Scientific) and 3.5 μL 1 M dithiothreitol were added. The mixture was immediately lysed by passing through a chilled French Press cell twice at 138 MPa. Unbroken cells and cell debris were removed from the lysate by centrifugation (22,000×g for 20 minutes at 4° C.). The clear supernatant was designated as cell extracts.

Enzyme activity assays. XR activity assay was carried out at 30° C. in 50 mM KPi buffer (pH 6) containing an appropriate amount of cell extracts, 200 mM D-xylose, and 0.2 mM of NADPH. The reaction was initiated by the addition of NADPH to the reaction mixture. XR enzyme activity was determined by monitoring the decrease in absorbance at 340 nm (Δε₃₄₀=6,220 M⁻¹·cm⁻¹) due to NADPH consumption. One unit of XR activity was defined as the consumption of 1 μmole of NADPH per minute under the defined conditions.

Formate dehydrogenase (FDH) activity assay was carried out at 35° C. in 50 mM KPi buffer (pH 7.5) containing an appropriate amount of cell extracts, 100 mM ammonium formate, and 1.5 mM of NAD⁺. The reaction was initiated by the addition of ammonium formate to the reaction mixture. FDH enzyme activity was determined by monitoring the increase in absorbance at 340 nm (Δε₃₄₀=6,220 M⁻¹·cm⁻¹) due to NADH production. One unit of FDH activity was defined as the production of 1 μmole of NADH per min under the defined conditions.

Glucose dehydrogenase (GDH) activity assay was carried out at 30° C. in 50 mM KPi buffer (pH 7.5) containing an appropriate amount of cell extracts, 100 mM glucose, and 1 mM of NAD⁺. The reaction was initiated by the addition of glucose to the reaction mixture. GDH enzyme activity was determined by monitoring the increase in absorbance at 340 nm (Δε₃₄₀=6,220 M⁻¹·cm⁻¹) due to NADH production. One unit of GDH activity was defined as the production of 1 μmole of NADH per minute under the defined conditions.

Spray-drying of recombinant P. pastoris cells. Since spray-drying required larger amount of biomass, 2-2.5 L of recombinant P. pastoris cells were cultivated. First, 50 mL BMGY cultures of selected recombinant strains were grown at 30° C. for 16 hours with orbital shaking at 300 rpm. After that, the 50 mL BMGY cultures were used to inoculate 250 mL BMGY media in 1-L baffled flasks and incubated at 30° C. for 24 hours. The 250 mL BMGY cultures were then used to inoculate 4-5 flasks 500 mL BMMY media in 2.8-L baffled flasks. These BMMY cultures were incubated at grown at 30° C. for 48 hours with orbital shaking at 300 rpm. At 24 hours, 0.5% methanol was added to keep the cultures induced. After 48 hours of growth in BMMY, the cells were harvested by centrifugation at 4,000×g for 10 minutes and washed once with phosphate buffered saline. The cell pellets were stored at −80° C. till spray-drying.

A Buchi B-190 spray-dryer with a pneumatic nozzle cleaner was used for all spray-drying procedures. Controlled operating parameters included: feed concentration, feed rate, air flow, temperature, and vacuum. For all experiments, the air flow was set to 700 L/hr and the vacuum was constant at −50 mbar. Experimental feed concentrations ranged from 30 to 600 mg/mL, feed rates spanned from 5 to 15 mL/minute, and operating temperatures were tested from 120 to 195° C. Selected recombinant Pichia pastoris cells (single and double recombinants) were grown in BMMY medium for 48 hours and induced for protein expression. The cells were collected by centrifugation (4,000×g for 10 minutes), washed once by phosphate-buffered saline, and stored at −80° C. Before spray-drying, the cells were removed from the freezer, allowed to thaw at room temperature, and excess water was removed by blotting the cells on filter paper. The blotted cells were weighed into several containers to yield desired cell concentrations after dilution to a fixed volume. The blotted cells were diluted to 100 mL total volume with deionized water. The containers were agitated forming cell suspensions at specified concentrations to be used as feeds to the spray-dryer.

Air flow was initiated to the spray-dryer at 700 L/hr, the aspirator was set to −50 mbar, and the heater was powered on. Deionized water was pumped through the spray nozzle in place of the cell suspension. The feed rate was set to the desired experimental value, and the heater was adjusted to yield the correct operating temperature. After reaching temperature equilibrium, the deionized water feed was replaced by the specified cell suspension. The spray-drying process lasted between 6 and 20 minutes based on the feed rate used. When the 100 mL cell suspension had been processed, the feed pump and heater were stopped, and air flow was allowed to cool the machine to 70° C. Then, the aspirator and air supply were shut down. The collection vessel was removed, and the P. pastoris cells were transferred to a scintillation vial. These vials were stored at ambient temperature. The feed line was flushed with deionized water and the glassware rinsed before spray-drying at other conditions.

Biotransformation of D-xylose to xylitol by spray-dried cells. Generally, D-xylose-to-xylitol biotransformation reaction contained 10 mg/mL spray-dried cells in 50 mM KPi (pH 7) buffer in a total volume of 5 mL. The reaction also contained 200 mM D-xylose and 0.25 mM NAD⁺. Reactions were incubated at 30° C. In some reactions, glucose and formate were added. Reactions were scaled up 10-fold in recycling experiments. After each round of reaction, spray-dried cells were collected by centrifugation (4,000×g, 4 minutes) and then re-suspended in fresh reaction solution for the next cycle of biotransformation.

The spray-dried cells were also tested for their capability to transform D-xylose in hemicelluloses hydrolysate to xylitol. Reactions ran with hemicelluloses hydrolysate contained 4.5 mL hydrolysate solution, 10 mg/mL spray-dried cells, and 0.25 mM NAD⁺. Final volume of the reactions were adjusted to 5 mL with deionized water. Glucose and formate were added to some reactions but only where indicated. Also, some of the reactions were run with hemicelluloses hydrolysate spiked with D-xylose to achieve higher final concentration of D-xylose. In those reactions, a pre-calculated amount of D-xylose crystals were directly added to the reactions to achieve the desired D-xylose concentrations.

All biotransformation reactions were incubated at 30° C. Aliquots reaction mixtures were removed from the reactions at various time points. Solids in the samples were removed by centrifugation at 13,000 rpm for 2 minutes, followed by filtration through a 0.22-μm filter. The solid-free supernatants were analyzed by for xylitol production and D-xylose consumption by a high performance liquid chromatography (HPLC) system.

Analytical procedures. Identification and quantification of D-xylose and xylitol were conducted with a Shimadzu LC-10AD HPLC system equipped with a photodiode array detector and a Shimadzu RID-10A refractive index detector. Separation of compounds was achieved on an Aminex HPX-87H column (Bio-Rad, 300×7.8 mm) The column was maintained at 30° C. during operation. Sulfuric acid (5 mM) was used as a mobile phase with a flow rate of 0.6 mL/minute.

### Results and Discussion

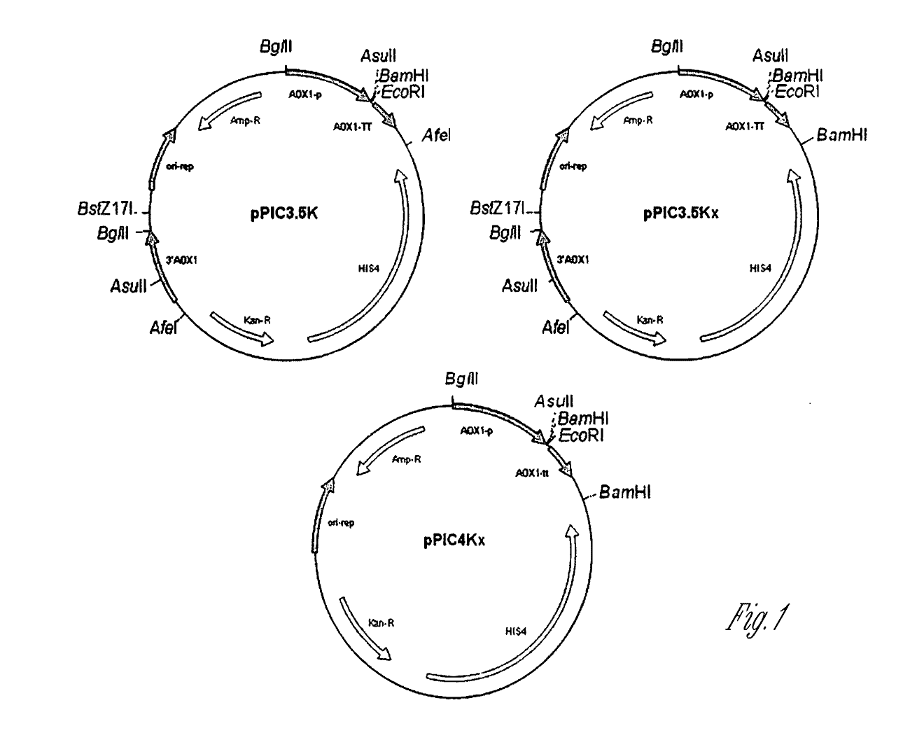

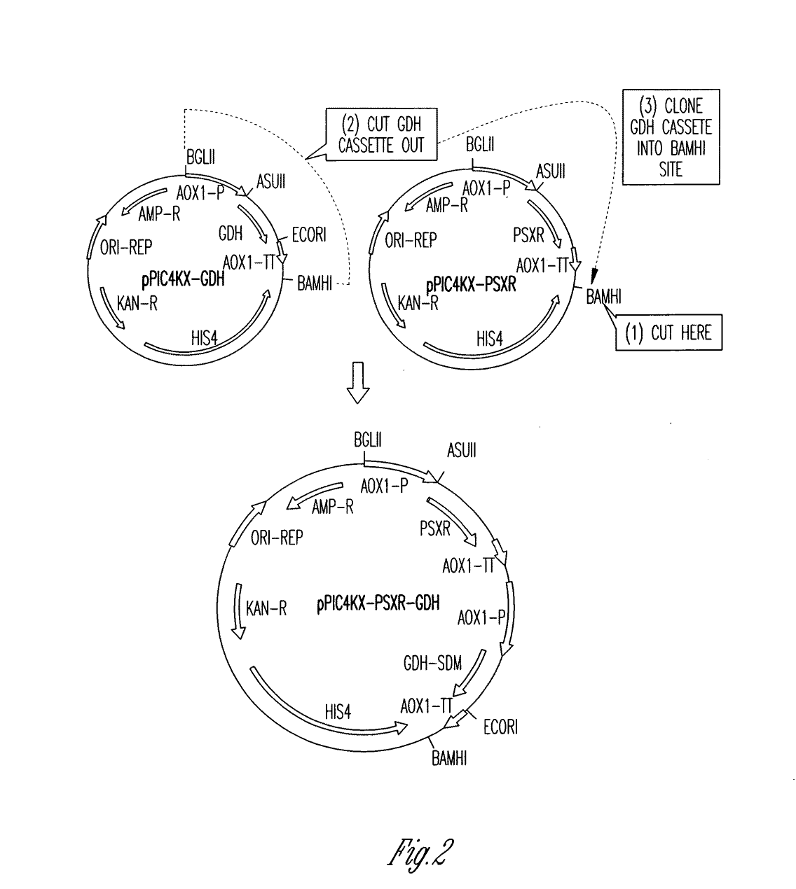

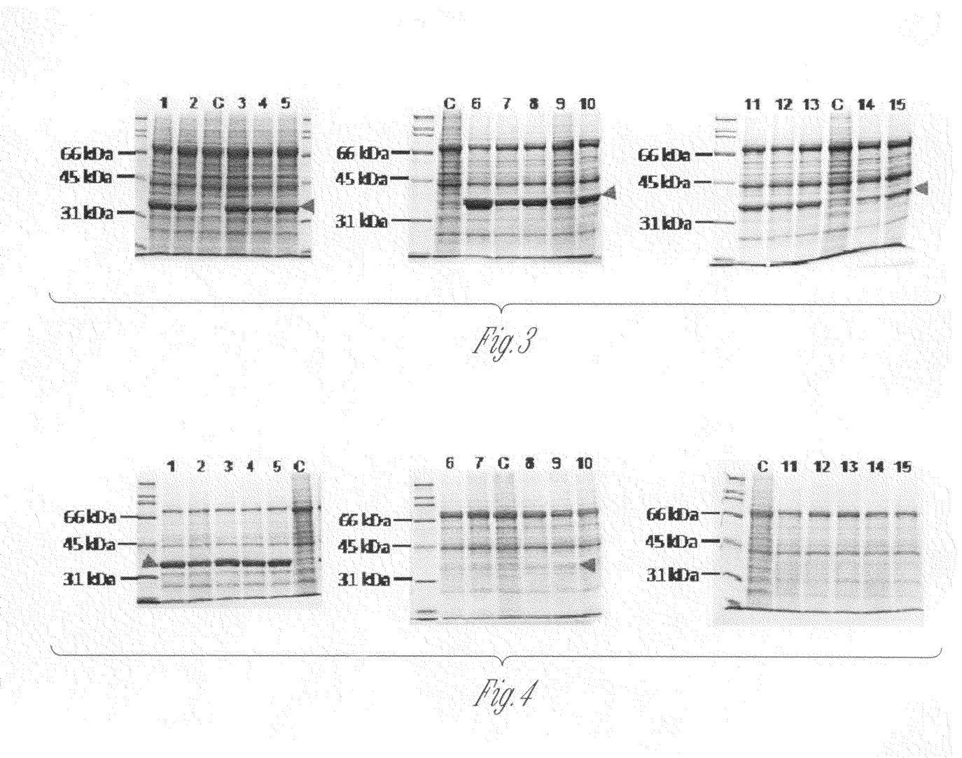

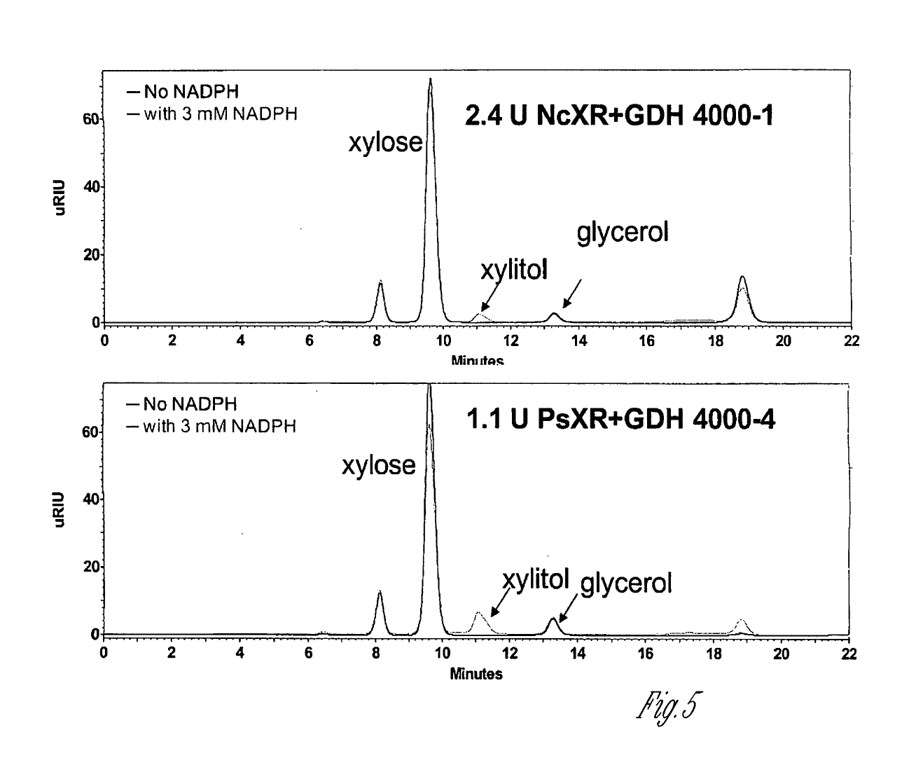

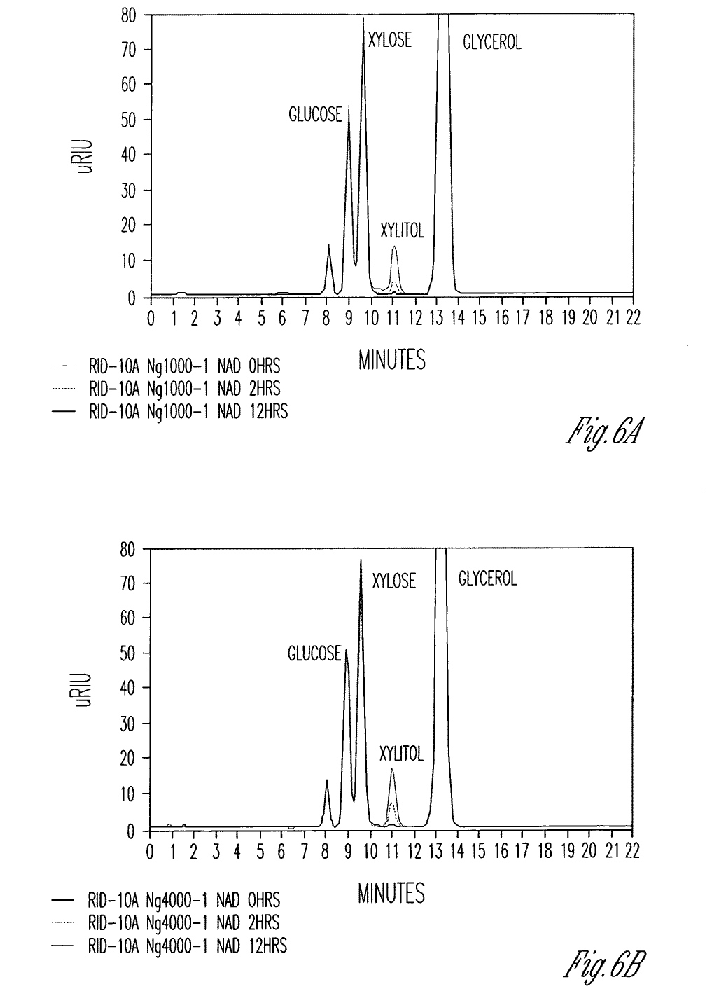

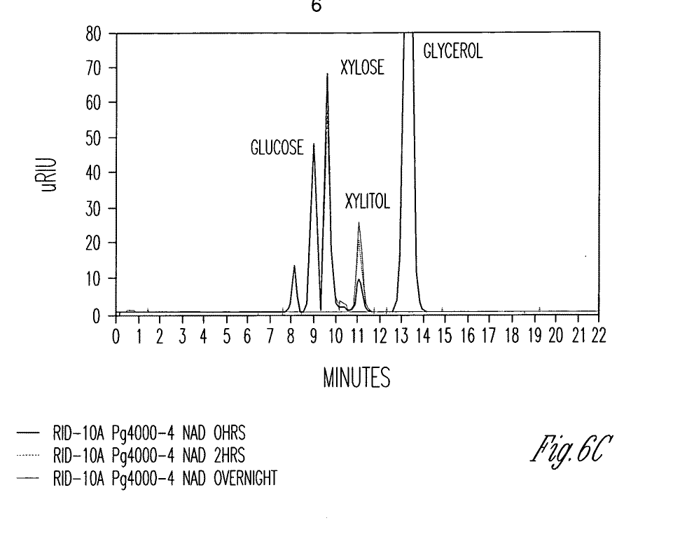

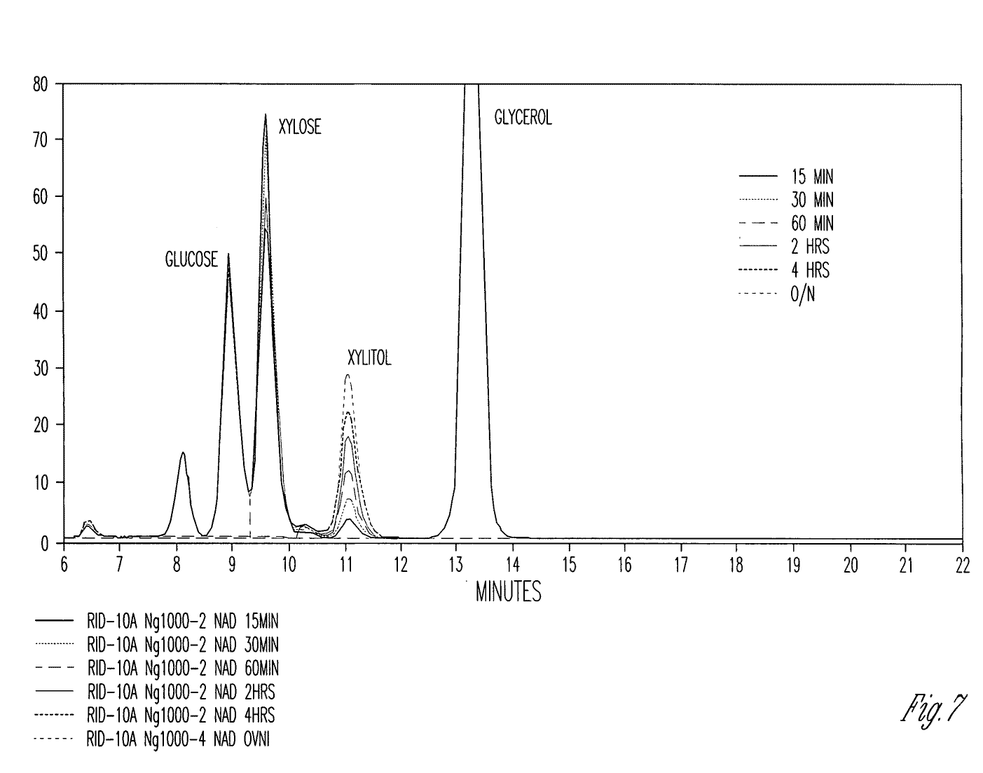

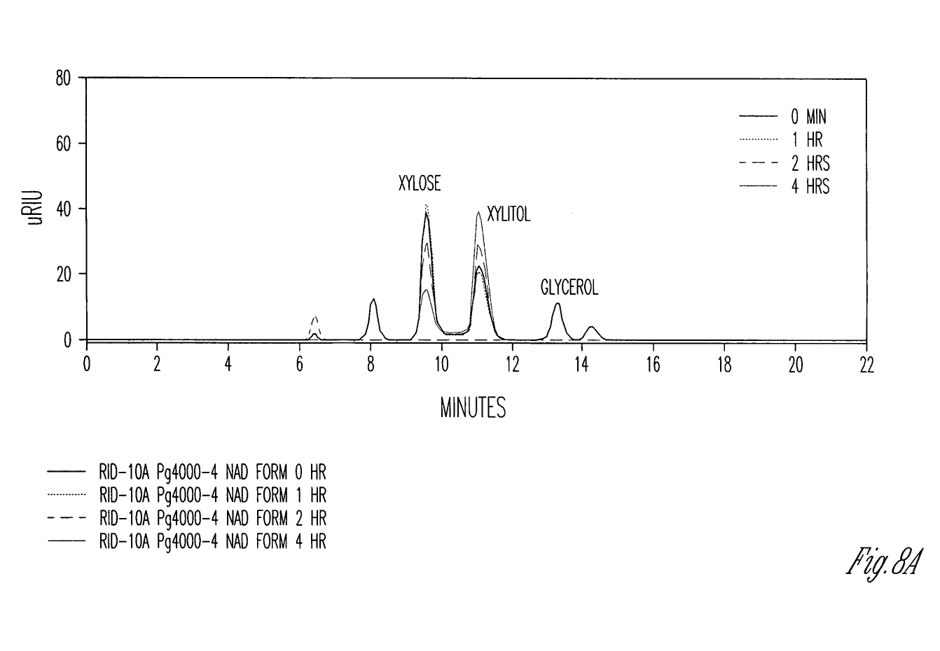

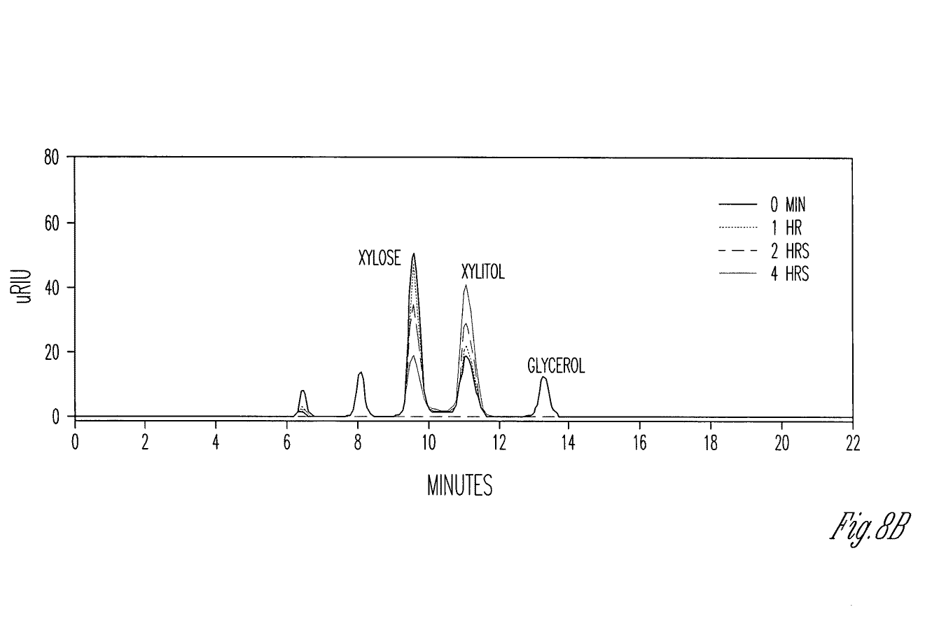

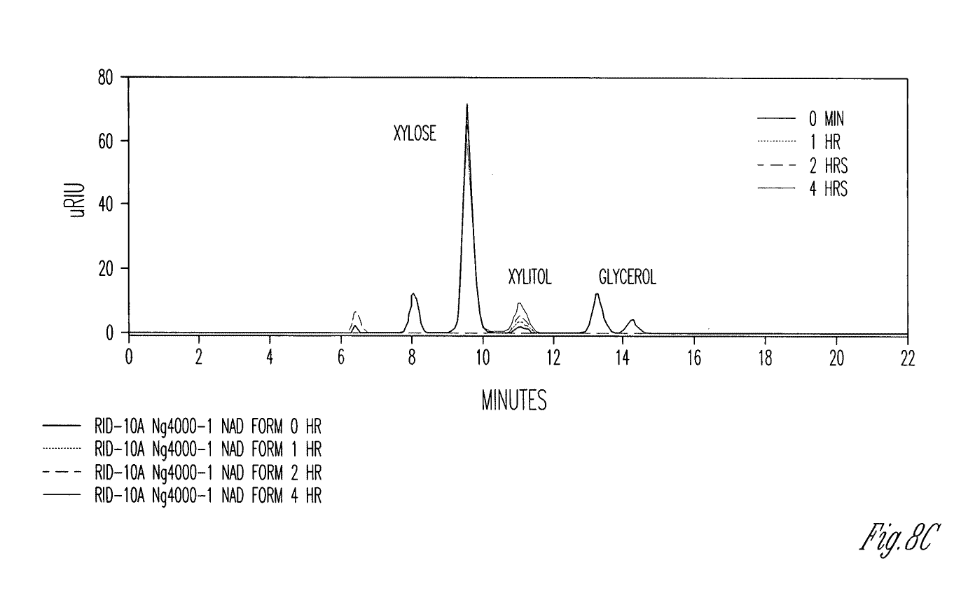

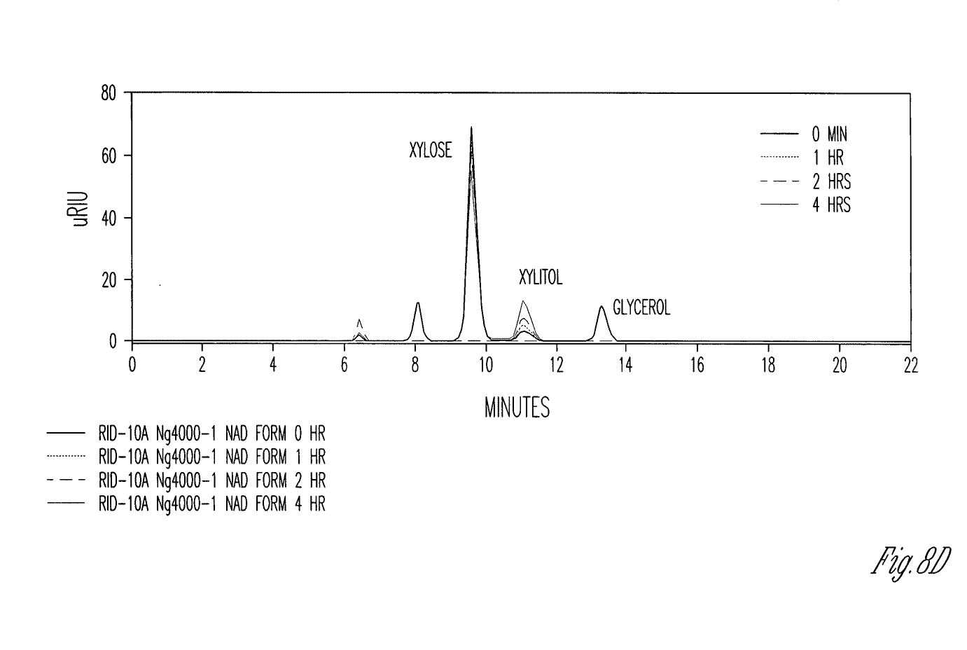

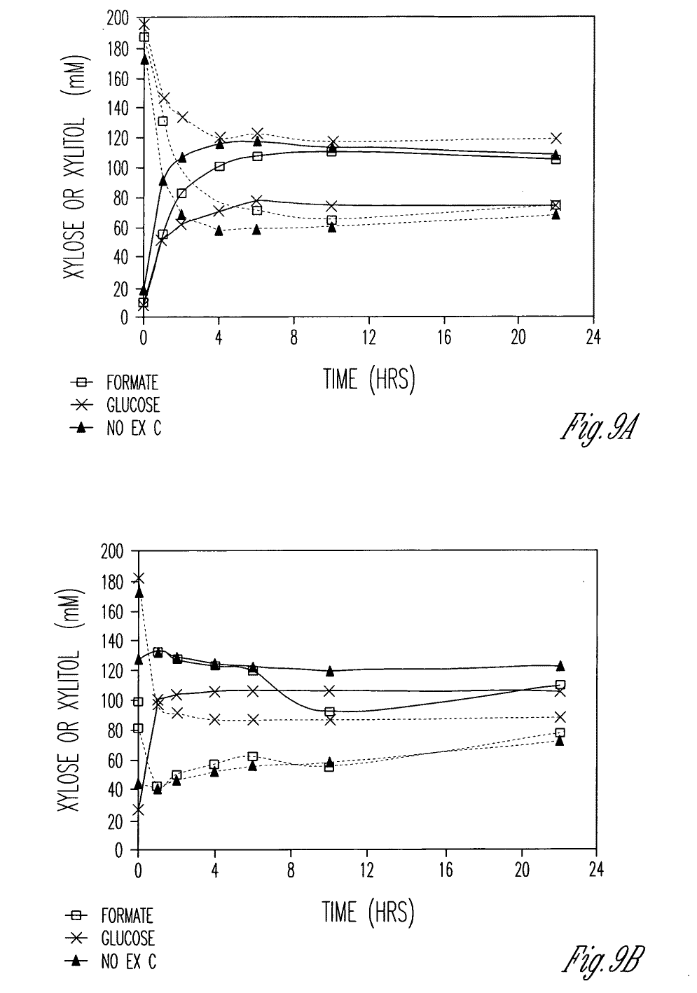

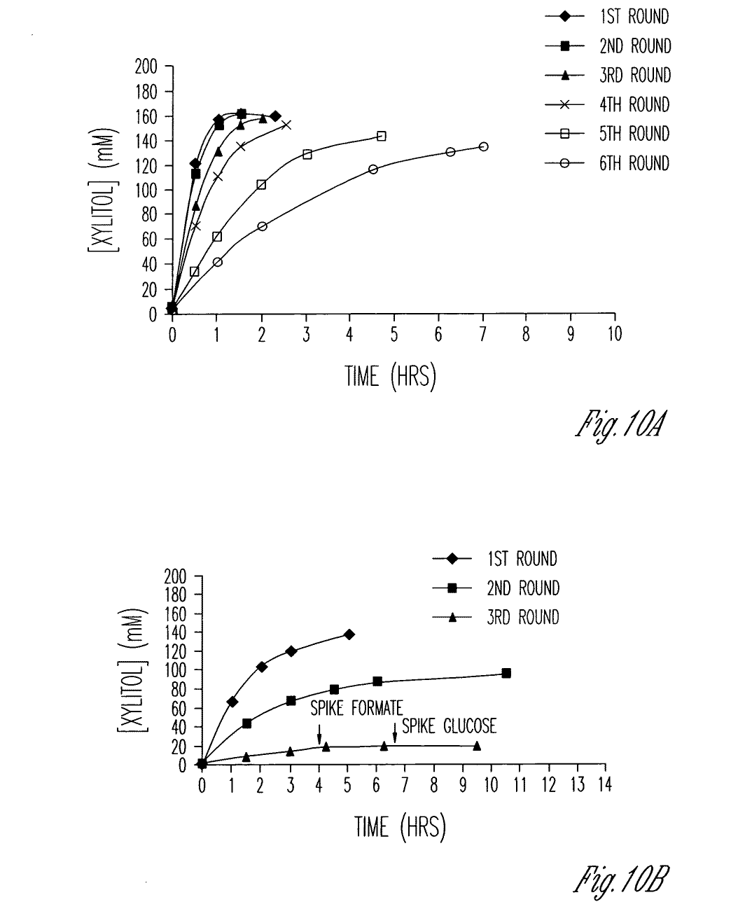

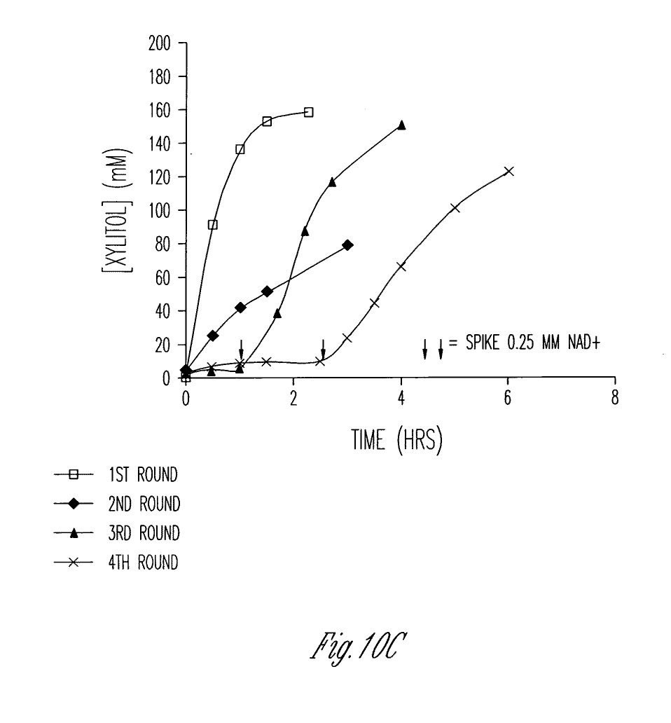

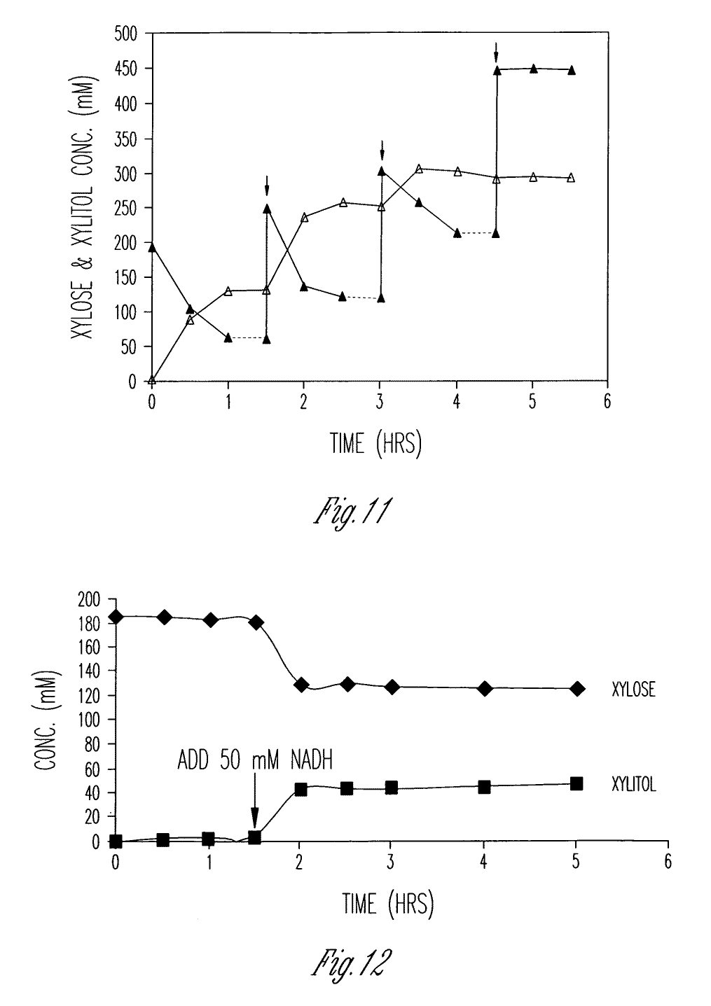

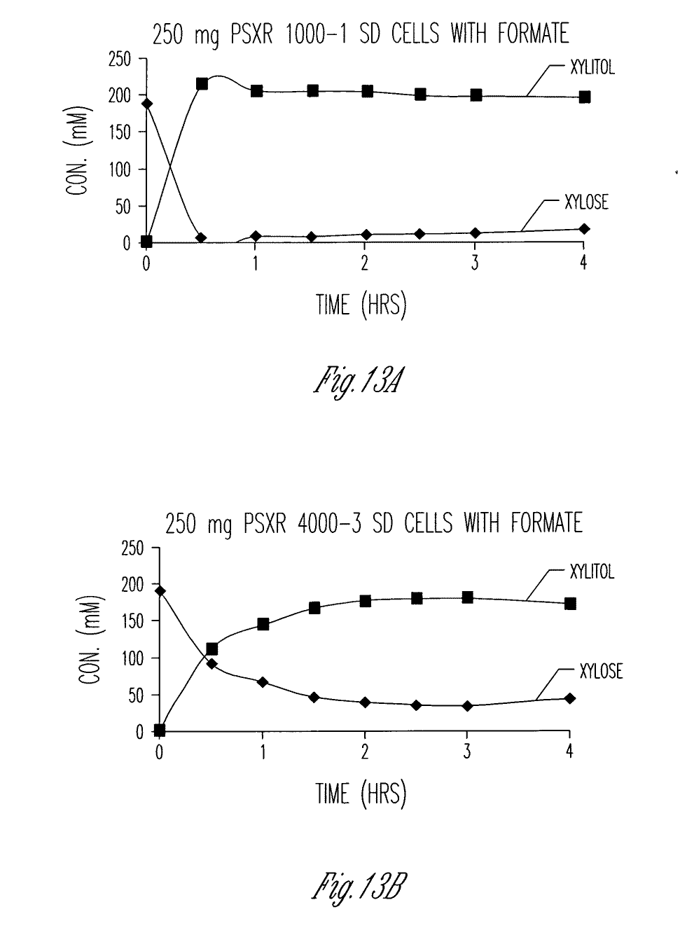

Synthesis of P. pastoris expression plasmid pPIC4Kx. P. pastoris expression plasmid pPIC3.5K (FIG. 1) was purchased from Invitrogen. This plasmid contains too many restriction enzyme recognition/cut sites which complicate cloning. Therefore, modifications of pPIC3.5K are necessary. First, an AfeI site on pPIC3.5K was removed by site-directed mutagenesis, creating plasmid pPIC3.5Kx (FIG. 1). During the same mutagenesis procedure, a new BamHI site was added to pPIC3.5Kx. This BamHI site is necessary for the construction of double recombinant strains in subsequent experiments (see below). After removal of the AfeI site, a 1093-bp DNA fragment was removed from pPIC3.5Kx by AfeI and BstZ171 digestion. This results in expression plasmid pPIC4Kx (FIG. 1), which contains a single Asull site for cloning of XR and gdh genes, and a single Bg/II site.

Cloning of XR and gdh gene into pPIC4Kx. Constructions of the XR genes expression plasmids pPIC4Kx-PsXR, pPIC4Kx-CpXR, and pPIC4Kx-NcXR and the gdh expression plasmid pPIC4Kx-gdh-sdm are described in the Materials and Methods section. Since the XR genes and gdh were cloned at the AsuII and EcoRI sites on pPIC4Kx, the BamHI site located between these 2 restriction enzyme cut sites (see FIG. 1) was removed from all of the resultant plasmid constructs. Hence, all of the XR and gdh expression plasmids had only a single BamHI site (in red, FIG. 1). This feature is important for the construction of the series of expression plasmids that expressed both XR and gdh.

To create expression plasmids that expressed both XR and gdh, the gdh expression cassette (“AOX1 promoter”+gdh+“AOX1 transcription terminator”) was removed from pPIC4Kx-gdh-sdm. This expression cassette was then cloned into the unique BamHI site of pPIC4Kx-PsXR, resulting in pPIC4Kx-PsXR-gdh. This cloning procedure is depicted in FIG. 2. Expression plasmids pPIC4Kx-CpXR-gdh and pPIC4Kx-gdh-NcXR were constructed similarly.

Transformation of P. pastoris GS115. After construction of all single recombinant plasmids (XR gene alone) and double recombinant plasmids (contained both XR and gdh), all plasmids were linearized by restriction enzyme BspEI, which cut these plasmids once within the HIS4 gene on the plasmid backbone. The linearized plasmids were used for transformation of P. pastoris GS 115 by electroporation. Transformants were selected based on their capability to grow on a mineral medium without the need of histidine supplementation, since strain GS115 has a His⁻ phenotype but a successful transformant would contain the HIS4 gene and became His⁺ phenotypically. In addition, since all of the expression plasmids could not replicate autonomously in P. pastoris, the recovery of His⁺ transformants indicates integration of the whole expression plasmid into P. pastoris GS115 genome. Integration probably occurred at the HIS4 locus of GS115 genome because the plasmids were linearized within the HIS4 gene before electroporation. Integration of the expression plasmids into P. pastoris genome allowed stable maintenance of the XR and gdh genes inside P. pastoris without the need of using any antibiotics.

Selection of His⁺ GS115 transformants. More than 500 His⁺ transformants were recovered from the transformation of each expression plasmids. Some of these transformants are expected to contain multiple copies of the expression plasmid since multiple integration events happen naturally in P. pastoris. These high-copy number transformants could produce higher levels of XR and GDH. Therefore, a screening procedure was used to screen for these high-copy number transformants, based on their resistance to geneticin. After the screening, numerous transformants were identified that were resistant to geneticin from 1 to 4 mg/mL. Therefore, 5 transformants originated from each expression plasmid were randomly picked: 2 were from an agar plate with 1 mg/mL geneticin and 3 came from an agar plate with 4 mg/mL geneticin. These transformants were cultured and induced for protein over-expression for 48 hours. After that, the cells were lysed and assayed for enzyme production by SDS-PAGE gels and enzyme activity assays.

XR and gdh expression studies. After culturing the selected transformants and induced for protein production by methanol, the cell were harvested and lysed. Cell lysates contained 15 μg of protein were loaded on SDS-PAGE gels (FIGS. 3 and 4).

The 3 XR genes expressed well in all the selected single recombinant clones (FIG. 3). Based on the intensity of the band, the NcXR gene was expressed at the highest level in the single recombinant clones, followed by the PsXR gene, and the CpXR genes. Among the five NcXR single recombinant clones, clone 1000-1 (FIG. 3, lane 6) had the highest level of NcXR protein. Co-expression of gdh led to lower expression level of XR in the double recombinant clones (FIG. 4). NcXR protein levels in double recombinant clones (FIG. 4, lanes 1-5) were significantly lower than single recombinant clones (FIG. 3, lanes 6-10). PsXR protein bands were barely visible in cell extracts prepared from PsXR+gdh double recombinants (FIG. 4, lanes 6-10), while a distinct CpXR band was not observed in cell extracts prepared from CpXR+gdh double recombinants (FIG. 4, lanes 11-15). Additionally, a distinct 28-kDa band which corresponded to the GDH protein was not observed in the cell extracts of all double recombinant clones.

Two clones from each group of recombinant culture were further analyzed for XR, GDH, and FDH activities (Table 3). The results showed that the 3 XR genes were expressed well in P. pastoris, consistent with the results from SDS-PAGE analyses in FIGS. 3 and 4. Expression levels of XR in these clones are comparable to those reported in literature. For example, C. guilliermondii XR was expressed in P. pastoris at 0.65 U/mg protein (Handumrongkul et al., 1998). C. shehatae, C. tropicalis, and C. tenuis XRs were expressed in E. coli at 13.5, 7.8, and 1.8 U/mg, respectively (Wang et al., 2007; Suzuki et al, 1999; Hacker et al., 1999). The NcXR 1000-1 single recombinant clone had 62 U/mg XR activity, exceeding the expression level of XR in previous reports. These results also showed that there were detectable GDH activities in the cell extracts prepared from double recombinant clones in spite of the absence of distinct GDH protein bands on SDS-PAGE gels (FIG. 4).

TABLE 3
Specific XR, FDH, and GDH activities in cell extracts
of selected single recombinant clones (with XR gene)
and double recombinant clones (with both XR and gdh).

| | Specific activity (U/mg protein) | | |
|---|---|---|---|
| Clone | XR | GDH | FDH |
| NcXR 1000-1 | 62.1 ± 7.1 | Not tested | 0.21 ± 0.03 |
| NcXR4000-2 | 35.7 ± 4.0 | Not tested | 0.29 ± 0.03 |
| PsXR 1000-1 | 10.1 ± 0.7 | Not tested | 0.34 ± 0.02 |
| PsXR 4000-3 | 9.7 ± 1.0 | Not tested | 0.31 ± 0.02 |
| CpXR 1000-6 | 1.3 ± 0.02 | Not tested | Not tested |
| CpXR 4000-8 | 3.3 ± 0.4 | Not tested | Not tested |
| NcXR + GDH 1000-2 | 22.4 ± 0.8 | 6.7 ± 0.5 | Not tested |
| NcXR + GDH 4000-1 | 38.6 ± 1.9 | 9.2 ± 0.3 | Not tested |
| PsXR + GDH 1000-8 | 1.7 ± 0.2 | 4.4 ± 0.6 | Not tested |
| PsXR + GDH 4000-4 | 10.2 ± 2.1 | 17.4 ± 1.0 | Not tested |
| CpXR + GDH 1000-5 | 0.0 | 4.6 ± 0.3 | Not tested |
| CpXR + GDH 4000-6 | 0.0 | 9.4 ± 0.8 | Not tested |
| pPIC4Kx empty | 0.2 ± 0.1 | 0.02 ± 0.01 | Not tested |
| vector control | | | |

FDH activities of 0.2-0.3 U/mg were detected in the cell extracts of single recombinant clones (Table 3). This FDH activity originated from P. pastoris formate dehydrogenase which is naturally produced when P. pastoris is grown on methanol. The detected level of FDH activities in the single recombinant clones are comparable to values reported in literature (Hou et al., 1982). However, the detected FDH specific activity was low compared to XR specific activity and might be insufficient to generate enough NADH to sustain the XR reaction. Three clones were chosen for further study: NcXR+GDH 1000-2, NcXR+GDH 4000-1, and PsXR+GDH 4000-4. These 3 clones were chosen because of their high levels of XR and GDH specific activities.

Biotransformation of D-xylose to xylitol by cell extracts of double recombinant clones. Cell extracts prepared from NcXR+GDH 4000-1 or PsXR+GDH 4000-4 ere incubated with 200 mM D-xylose and 0.2 mM NADPH in 50 mM KPi buffer (pH 6.0). Oxidation of NADPH was monitored spectrophotometrically at 340 nm. When NADPH was consumed, a new aliquot of NADPH was added to the reaction mixture. This cycle was repeated until a total 3 mM of NADPH was consumed. The reaction mixture was then analyzed by HPLC for xylitol (FIG. 5). A small xylitol peak was detected when D-xylose was incubated with either of the cell extracts. Production of xylitol was NADPH-dependent; omission of NADPH from the reaction mixture resulted in no xylitol production (black trace in the 2 chromatograms). The results indicate the XR expressed in P. pastoris is active and can transform D-xylose to xylitol.

The 2 reactions that demonstrated transformation of D-xylose to xylitol by addition of NADPH (FIG. 5) were limited by the low concentration of NADPH. Therefore, enzyme reactions were set up at 30° C. with cell extracts prepared from double recombinant clones with 200 mM D-xylose, 100 mM glucose, and 0.25 mM NAD⁺ in 50 mM KPi (pH 7.0) buffer with 10% glycerol for stabilizing enzymes. Since GDH activities were detected in these cell extracts (Table 3), oxidation of glucose by GDH should reduce NAD⁺ to NADH, which would in turn be utilized by XR for xylitol production. HPLC analyses of the enzyme reactions demonstrated xylitol production using cell extracts prepared from NcXR+GDH 1000-2, NcXR+GDH 4000-1, and PsXR+GDH 4000-4 (FIG. 6). The PsXR+GDH 4000-4 cell extracts appeared to be the most efficient in transforming D-xylose. Once D-xylose was mixed with this cell extract, xylitol was immediately produced and resulted in a xylitol peak in the 0 hour chromatogram (black trace in FIG. 6C). Reaction with PsXR+GDH 4000-4 cell extracts also had the lowest level of XR activity but produced the most xylitol with 12 hours. Despite continuous production of xylitol, glucose was not consumed in all three reactions in FIG. 6. This observation was reproduced in a similar reaction using 130 U of NcXR from NcXR+GDH 1000-2 cell extracts (FIG. 7).

Since FDH is a native enzyme in P. pastoris, it should be co-induced naturally in the double recombinant clones when those clones were grown in BMMY media. Therefore, it was tested whether oxidation of formate by the native FDH would couple with the cloned XR for xylitol production. Cell extracts prepared from double recombinant clones PsXR+GDH 4000-4 and NcXR+GDH 4000-1 were incubated at 30° C. with 200 mM D-xylose, 100 mM formate, and 0.25 mM NAD⁺ in 50 mM KPi (pH 7.0) buffer with 10% glycerol for stabilizing enzymes. Xylitol production was detected in reactions with both types of cell extracts (FIG. 8). Surprisingly, xylitol was also produced even the reaction contained no formate. Xylitol production was a biological reaction since boiled cell extracts did not catalyze transformation of D-xylose to xylitol. These results suggest the cell extracts contain another source of electron donor which reduces NAD⁺ to NADH for the XR reaction. Another important observation is that cell extracts prepared from clone PsXR+GDH 4000-4 were more efficient in transformation of D-xylose to xylitol (FIGS. 8A and B) even though the PsXR protein was not over-produced in the clone compared to the NcXR protein (FIG. 4). Xylitol was rapidly produced once the D-xylose was in contact with the cell extracts, resulting in the detection of xylitol in the time=0 sample (black traces in FIGS. 8A and B).

Biotransformation of D-xylose to xylitol by spray-dried double recombinant clones. Cultures of clones PsXR+GDH 4000-4 and NcXR+GDH 4000-1 were spray-dried after induction of protein expression in BMMY media, as described in Materials and Methods. The spray-dried cells were then tested for their capability for xylitol production at 30° C. by suspending in 50 mM KPi (pH 7.0) buffer with 200 mM D-xylose and 0.25 mM NAD⁺. Formate (100 mM), glucose (100 mM), or no additional carbon source was added to the cell suspension. The results showed that the spray-dried cells remained active and capable of transforming D-xylose to xylitol (FIG. 9). As observed in previous experiments using cell extracts, glucose or formate was not needed to sustain the XR reaction. In fact, addition of glucose to the reaction mixtures slightly inhibited XR reaction. Hence, there are sufficient electron donors inside the spray-dried cells which remain redox active and capable of reducing NAD⁺ to NADH for XR reaction. PsXR+GDH 4000-4 spray-dried cells were most active (FIG. 9B); xylitol was detected at the time=0 sample suggesting immediate transformation of D-xylose to xylitol once the spray-dried cells contacted the substrate. In the reaction using PsXR+GDH 4000-4 with no addition of carbon source, about 130 mM of xylitol was produced, indicating approximately a 65% conversion.

Recycling spray-dried double recombinant clones for xylitol production. The reusability of spray-dried PsXR+GDH 4000-4 and NcXR+GDH 4000-1 cells for multiple cycles of xylitol production was tested. The 50 mL reactions were set up in 50 mM KPi (pH 7.0) buffer with 10 mg/mL spray-dried cells, 200 mM D-xylose and 0.25 mM NAD⁺. Neither glucose nor formate was added. The results (FIG. 10) showed that PsXR+GDH 4000-4 spray-dried cells could be re-used for 6 cycles but NcXR+GDH 4000-1 spray-dried cells could be re-used for only 2 cycles. After both types of spray-dried cells lost activities, addition of either glucose or formate did not restore activities (FIG. 10B). In addition, PsXR+GDH 4000-4 spray-dried cells were found to sustain 2 cycles of reaction without the need of an external source of NAD⁺ (FIG. 10C). After 2 cycles of reactions without NAD⁺, the cells displayed no activity unless NAD⁺ was added. Similar results were obtained with respect to NADP⁺. The results suggest NAD⁺ inside the spray-dried cells leached out completely while washing during 2 cycles of reactions. Interestingly, sufficient amount of the unknown electron donor(s) remained inside the cells to reduce the exogenously added NAD⁺ to NADH after 2 cycles.

Repeat spiking of D-xylose. Using spray-dried PsXR+GOH 4000-4 cells, about 80% conversion of 200 mM D-xylose to xylitol was observed (FIGS. 10A and C). The lack of conversion beyond 80% could be due to inhibition by xylitol (e.g., product inhibition) or low concentration of D-xylose. To evaluate these 2 possibilities, a reaction was set up with spray-dried PsXR+GDH 4000-4 cells in 50 mM KPi (pH=7.0) buffer with 200 mM D-xylose. No NAD⁺ was added to the reaction. The progress of the biotransformation of D-xylose was monitored. Once the transformation stopped, a new aliquot of 200 mM D-xylose was added to the reaction. The data showed that the biotransformation reaction could be prolonged by adding D-xylose (FIG. 11). However, once the xylitol accumulated to above 300 mM, the reaction stopped in spite of the presence of high concentration of D-xylose. The data suggest the reaction does not proceed at low concentration of D-xylose but high concentration of xylitol could also inhibit the reaction. A reaction with 0.25 mM NAD⁺ added to the reaction produced similar results (data not shown).

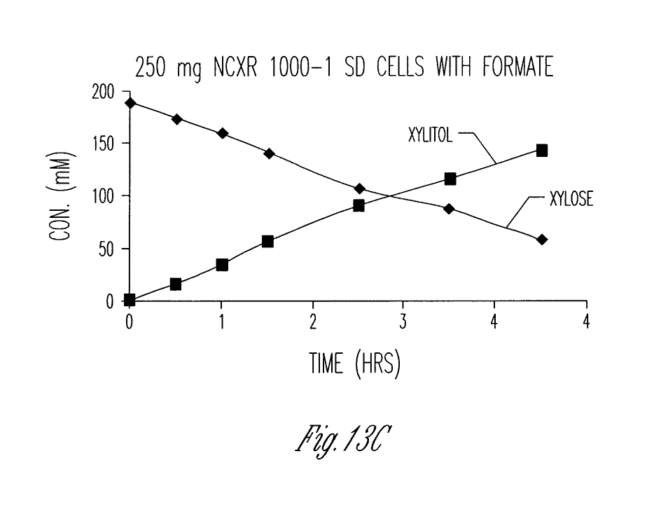

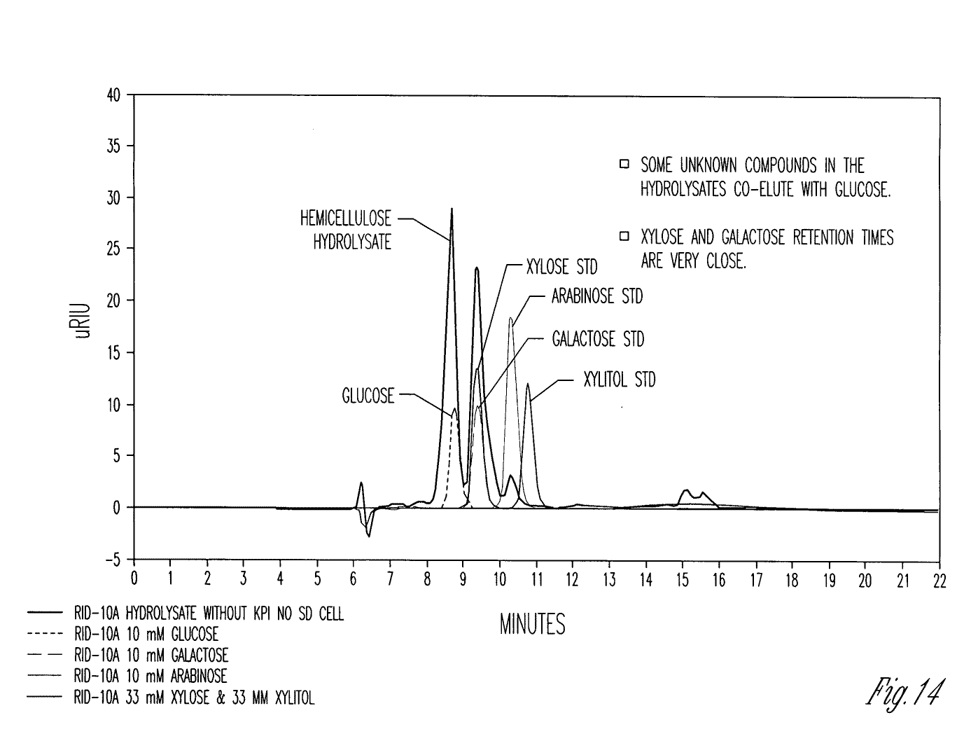

Biotransformation of D-xylose to xylitol by spray-dried single recombinant clones. Since biotransformation of D-xylose to xylitol did not require addition of external electron donor such as glucose or formate, the cloned gdh gene in all the double recombinant clones might not be essential. Therefore, single recombinant clones were tested for the biotransformation reaction. Three single recombinant clones (NcXR 1000-1, PsXR 1000-1, and PsXR 4000-3) were cultured in BMMY, induced for XR production, and then spray-dried. Strain NcXR 1000-1 was chosen because it had the highest level of expressed XR specific activity (Table 3). PsXR specific activity level in PsXR 1000-1 and PsXR 4000-3 was comparable to that in PsXR+GDH 4000-4 double recombinant clone (Table 3). A reaction was set up using PsXR 4000-3 spray-dried cells (10 mg/mL spray dried cells in 50 mM KPi (pH 7.0) buffer, with 200 mM D-xylose). Surprisingly, this reaction did not produce any xylitol. Increasing the spray-dried cell concentration to 50 mg/mL and addition of NAD⁺ (final concentration=0.25 mM) to the reaction was not helpful (data not shown). However, addition of NADH to a final concentration of 50 mM resulted in complete and stiochiometric production of xylitol from D-xylose (FIG. 12). The results indicate PsXR was active in the PsXR 4000-3spray-dried cells but the cells were limited in electron donor for the reduction of NAD⁺ to NADH. This conclusion was further supported by the fact that single recombinant spray-dried cells transformed D-xylose to xylitol when formate was included in the NAD⁺-free reaction mixtures (FIG. 13). Among the three selected single recombinant clones, PsXR 4000-3 appeared to be the best clone.

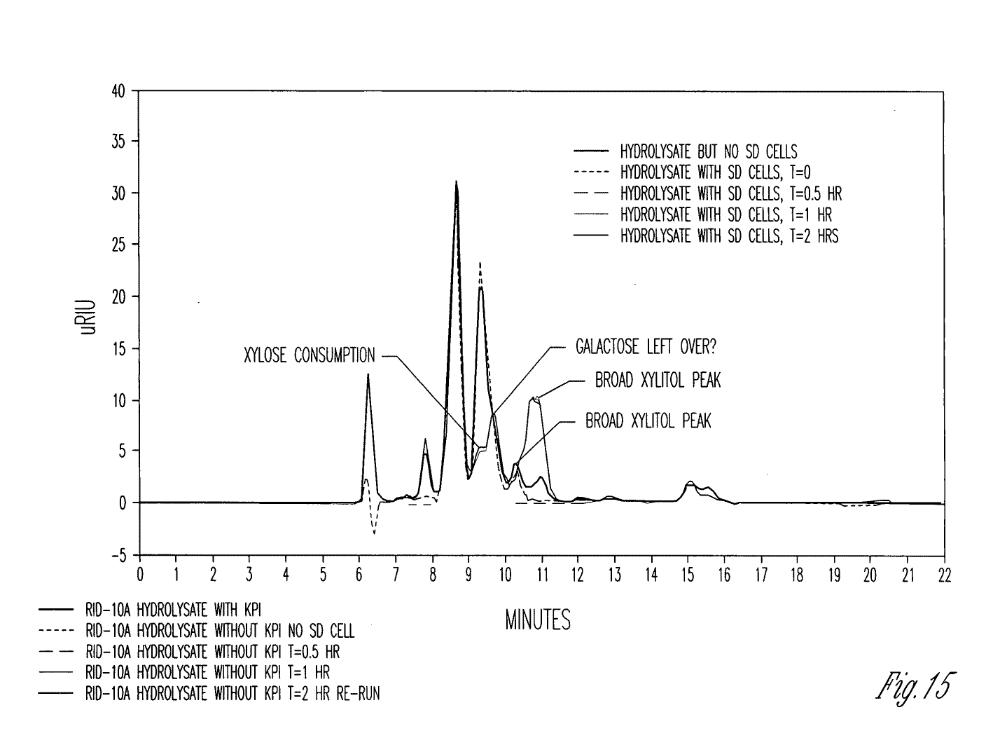

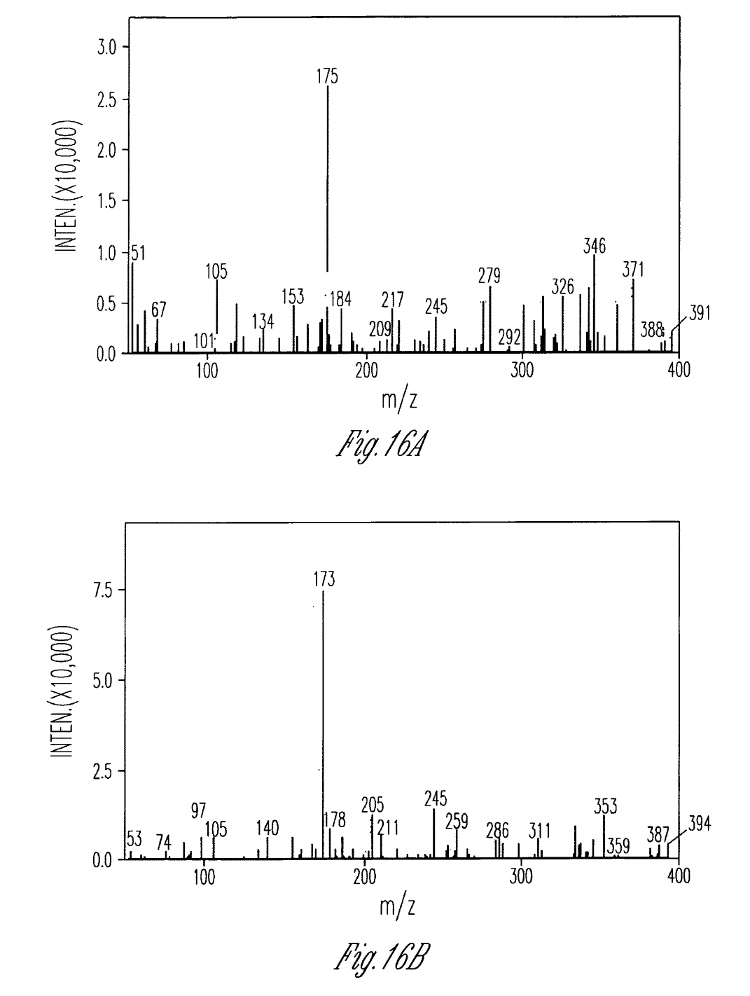

Biotransformation of D-xylose in hemicelluloses hydrolysates. A sample of hemicelluloses hydrolysate had a pH value of 7.0. HPLC analyses confirmed approximately 50-60 mM D-xylose in the hydrolysate (FIG. 14). A large amount of glucose was detected.

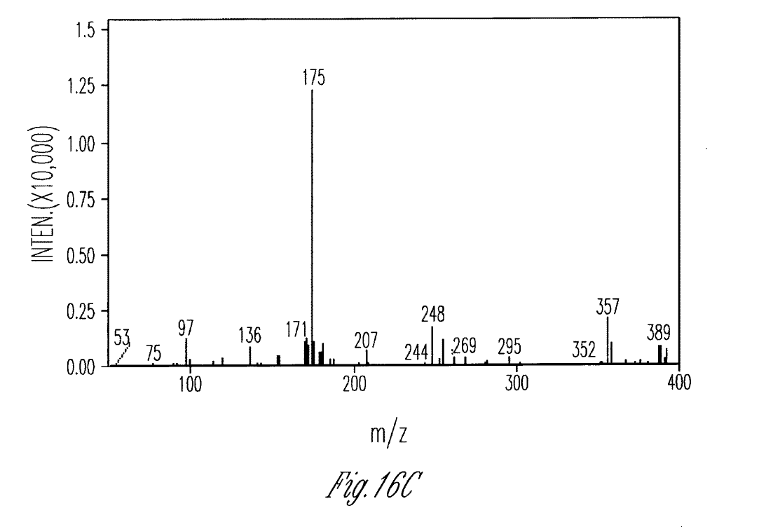

Spray-dried double recombinant PsXR+GDH 4000-4 cells were tested could transform the D-xylose in the hydrolysate to xylitol. Because of the low concentration of D-xylose in the hydrolysate, 50 mg/mL of spray-dried cells were used (e.g., 5-fold higher than regular reaction). The cells were incubated directly with the hydrolysate at 30° C. without the addition of KPi buffer, NAD⁺, glucose, or formate. Production of xylitol happened almost immediately and completed within 30 minutes (FIG. 15). A broad product peak with retention time very close to xylitol (about 10.9 minutes) was formed. This product was collected from the HPLC effluent stream and directly injected into an electron spray ionization-mass spectrometer (ESI-MS) operating in positive ion mode. ESI mass spectrum of this product had a base peak with m/z=175 (FIG. 16A). ESI mass spectra of standard xylose (MW=150) and xylitol (MW=152) had base peaks with m/z=173 and 175, respectively (FIGS. 16B and C). These molecular ions were not detected when deionized water was injected. The results suggest xylose and xylitol probably formed positively charged adducts with Na⁺ (MW=23), forming respective molecular ions with m/z=173 and 175. The results also confirm PsXR+GDH 4000-4 converted D-xylose in the hydrolysate into xylitol.

In previous experiments using pure D-xylose, higher concentrations of xylitol inhibited the XR reaction (FIG. 11). However, the effect of high initial concentration of D-xylose on XR was not examined. Consequently, various amounts of D-xylose were added to the hemicelluloses hydrolysate, creating hydrolysate solutions with 1,550, 800, 800, and 450 mM of D-xylose. These various hydrolysate solutions were then incubated with 3 different concentrations (10, 30, and 50 mg/mL) of spray-dried PsXR+GOH 4000-4 cells without KPi buffer, NAD⁺, glucose, or formate. The results are summarized in Table 4. No inhibition by a high concentration of D-xylose was observed since the initial reaction rates at different concentrations of D-xylose were similar (data not shown). The lower % conversion with high substrate concentration was due to xylitol inhibition. This product inhibition could be alleviated by increasing the amount of spray-dried cells in the reaction, as observed in the series of reactions using 50 mg/mL spray-dried cells. Moreover, note that the clone with the most favorable characteristics (PsXR+GDH cells) did not include a recombinant XR that was improved by directed evolution and had low activity in vitro.

TABLE 4
Percent conversion of D-xylose to xylitol by various
concentration of spray-dried PsXR + GDH cells.

% xylose converted to xylitol

| Xylose¹ | 10 mg/mL | 30 mg/mL | 50 mg/mL |
|---|---|---|---|
| conc.(mM) | SD cells | SD cells | SD cells |
| 1550 | 21 (10)² | 60 (4.5) | 72 (2.5) |
| 1050 | 32 (10) | 71 (3) | 76 (2) |
| 800 | 36 (10) | 74 (3) | 77 (1) |
| 450 | 50 (10) | Not tested | 78 (0.75) |

¹Total concentration of D-xylose after spiking (approximately 50 mM D-xylose originally present in hydrolysate).
²Number in parentheses = hours to achieve maximum conversion.

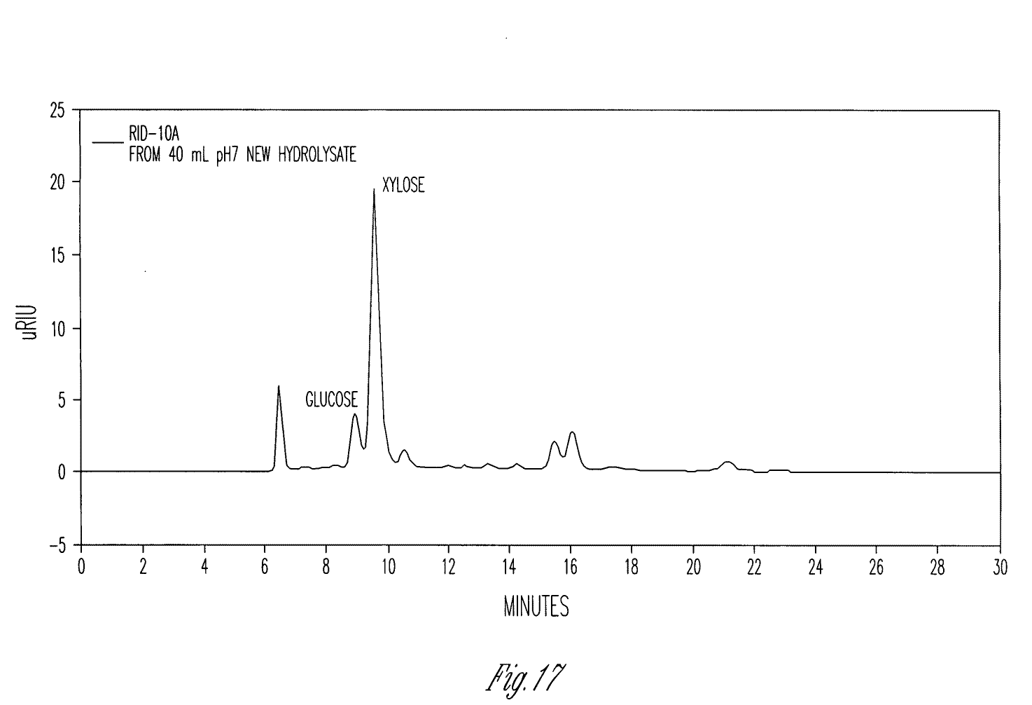

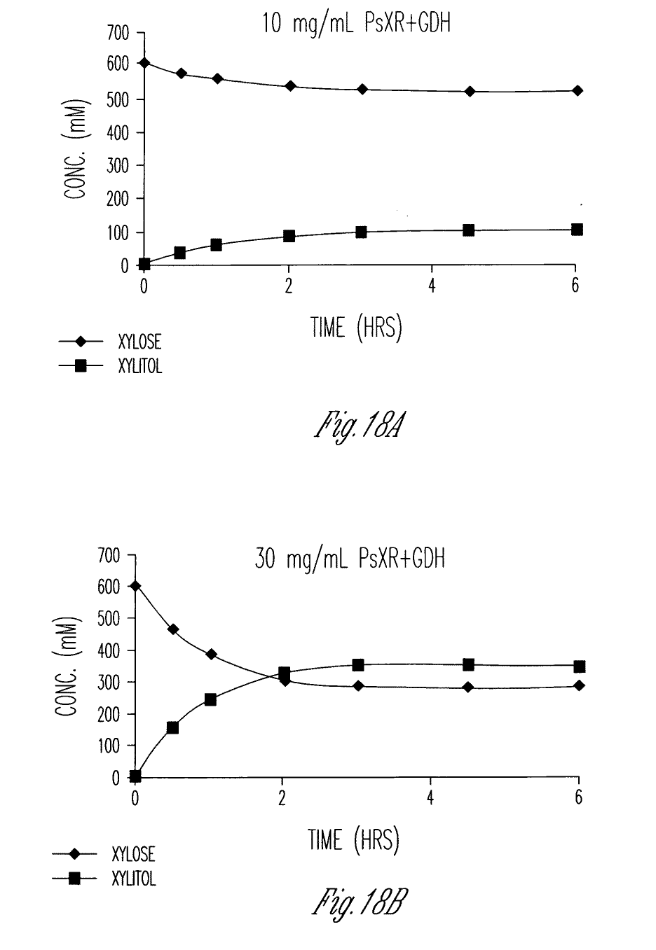

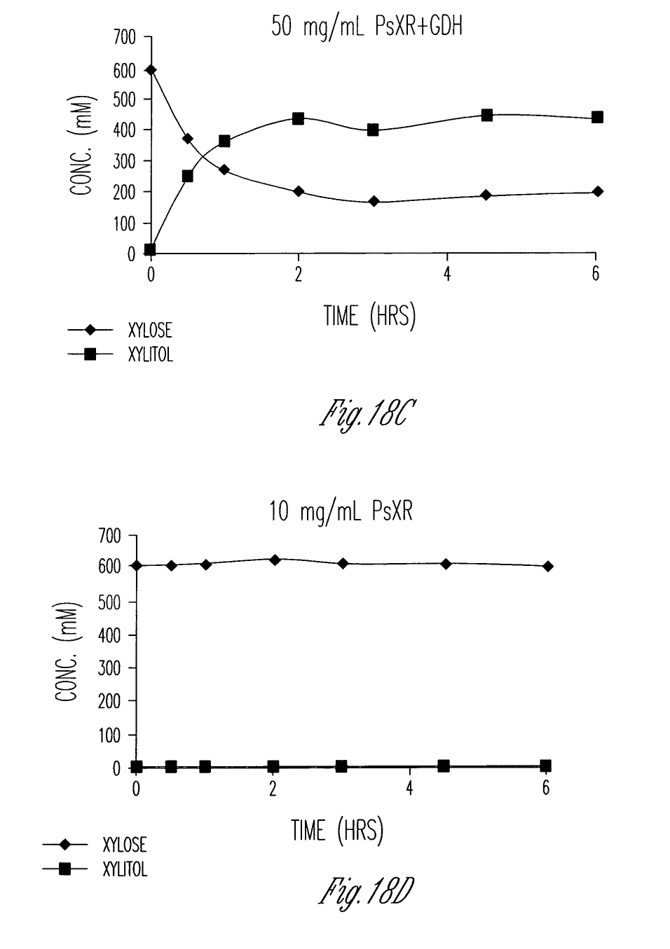

Another hydrolysate solution with a pH=3.8 was obtained and the pH adjusted to 7.0 by adding NaOH. HPLC analysis of the pH 7.0 hydrolysate (FIG. 17) showed that approximately 52 mM of D-xylose was present in this solution and it contained much less glucose than the previously tested hydrolysate solution (FIG. 14).

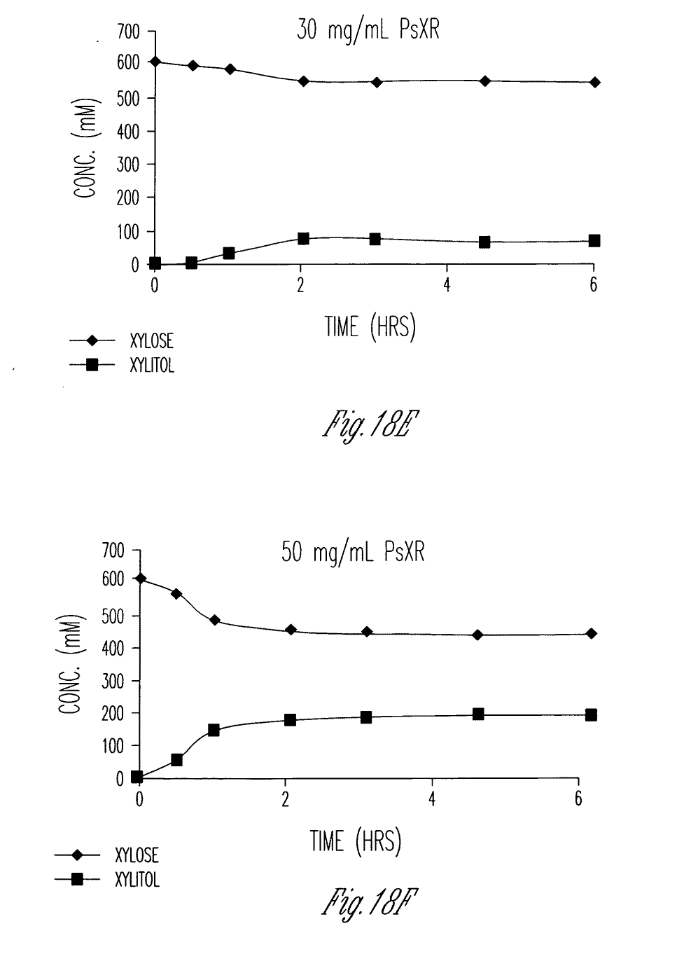

Using this hydrolysate solution the performance of spray-dried PsXR+GDH 4000-4 (double recombinant) was compared with PsXR 4000-3 (single recombinant) cells. Additional D-xylose was added to the hydrolysate to increase D-xylose concentration to 600 mM. The spiked hydrolysate solution was incubated with 10, 30, and 50 mg/mL of single or double recombinant spray-dried cells. Since the single recombinant PsXR 4000-3 required a source of electron donor, 600 mM formate was added to the reactions with PsXR 4000-3 cells. The pH values of all reactions were adjusted to pH 7.0 by the addition of NaOH. The results showed that the double-recombinant PsXR+GDH 4000-4 cells was a better clone than the single recombinant PsXR 4000-3 cells because the double recombinant spray-dried cells achieved higher % conversion with a lower concentration of spray-dried cells (FIG. 18). The maximum conversion (about 430 mM xylitol or 72%) was achieved by 50 mg/mL PsXR+GDH 4000-4 cells in 2 hours (FIG. 18C). The same level of PsXR+GDH 4000-4 cells only took approximately 1 hour to convert 77% of 800 mM D-xylose to xylitol (Table 4). In that reaction, D-xylose was spiked into the 1st batch of hydrolysate solution. The pH values of the reactions with PsXR+GDH 4000-4 spray-dried cells (double recombinant) decreased from 7 to approximately 5 in 1 hour after the start of the reactions. Meanwhile, pH values of the reactions with PsXR 4000-3 spray-dried cells (single recombinant) increased from 7 to approximately 8 two hours after the start of the reactions. Substantially similar results were observed when reactions were scaled up to 1 L.

In summary, three different XR genes were individually cloned into P. pastoris GS115, generating 3 single recombinant clones. A gdh gene was subsequently cloned into the 3 XR single recombinant clones, creating 3 double recombinant clones (with XR AND gdh genes). Expression of the XR and gdh genes in P. pastoris was confirmed by SDS-PAGE. Activities of the cloned genes were confirmed by measuring the enzyme activities in cell extracts, and productivity of xylitol from D-xylose.

Spray-dried double recombinant clones converted D-xylose to xylitol without the need for adding formate or glucose (e donor). The reactions could be sustained for 2 cycles without NAD⁺. Single recombinant clones also converted D-xylose to xylitol, but formate (e donor) was needed. Still, NAD⁺ was not required (when a catalytic amount of NAD is added to DH coupled reactions, it may be present in an amount of about 0.2 mM to about 7 mM). Thus, both types of clones do not require addition of NAD⁺. The data suggest that sufficient amounts of NAD⁺ are present in spray-dried cells and are redox active. Double recombinant clones appear to accumulate significant amount of e⁻ donor during cultivation for re-generating NADH. The nature of this e⁻ donor is unclear. Although reactions proceeded without NAD⁺ for 2 cycles, resulting in reactions that began very slowly in cycles 3 and 4 possibly due to NAD⁺ leaching out from SD cells, a NAD⁺ spike stimulated the reaction. Thus, NAD(P)⁺ addition is not required for at least a few cycles. Nevertheless, the rate of xylitol formation was very rapid and the reaction was specific as no activity was observed with L-arabinose (200 and 20 mM).

Both PsXR 4000-3 and PsXR+GDH 4000-4 were capable of converting D-xylose in corn stover hemicellulose hydrolysate solutions to xylitol. The addition of several additional components to the crude stover hydrolysate had no effect on the conversion of xylose to xylitol. When both clones were compared under identical conditions, the double recombinant PsXR+GDH 4000-4 performed better than PsXR 4000-3 in terms of percent conversion. PsXR+GDH 4000-4 produced approximately 430 mM xylitol (65 g xylitol/L) from 600 mM D-xylose (90 g/L) in 2 hours.

### EXAMPLE 3

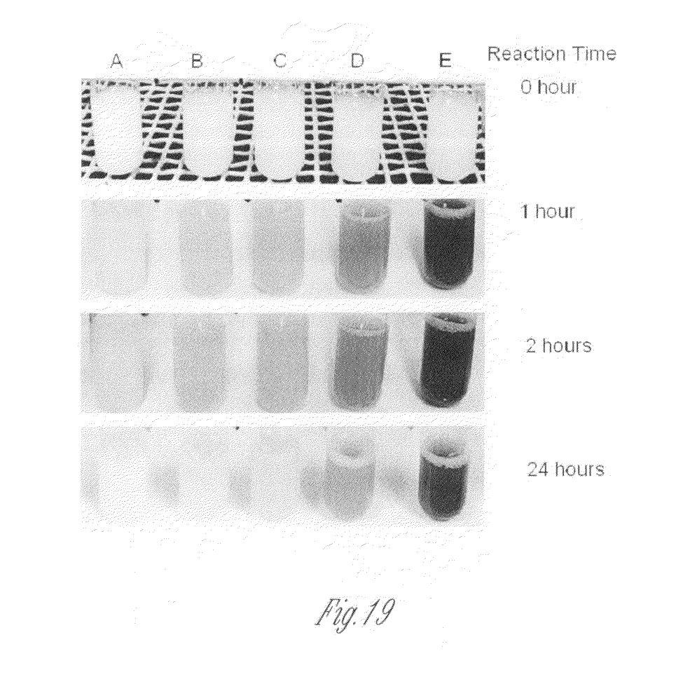

Other exemplary oxidoreductase reactions for biocatalyis using spray-dried cells include the conversion of L-sorbose by sorbose reductase, e.g., Candida sorbose reductase, to sorbitol, the conversion of mannose by mannitol dehydrogenase, e.g., celery mannitol reductase, to mannitol, the conversion of catechol by tyrosine phenol lyase (in the presence of pyruvate and NH₃)to L-DOPA, the conversion of tyrosine by tyrosine hydroxylase (in the presence of tetrahydrobiopterin) or by 4-hydroxyphenylacetate 3-hydoxylase (HpaB, a monooxygenase, and HpaC, an FADH₂ dependent oxidoreductase) to L-DOPA, the conversation of indole to indigo by naphthalene dioxygenase (see FIG. 19), and the reductive deamination of a substrate by phenylalanine dehydrogenase, e.g., from Thermoactinomyces intermedius, (in the presence of ammonium formate) to saxagliptin.

In one embodiment, a cytochrome P450 enzyme that metabolizes a particular drug, e.g. 2D6 or T. cuspidata cytochrome P450 taxoid 10beta-hydroxlyase, is expressed in conjunction with an oxidoreductase, such as a human NADPH P450 oxidoreductase or T. cuspidata NADPH P450 reductase, respectively, is expressed in microbial cells which are then spray-dried. In another embodiment, a soluble P450 bifunctional enzyme is employed, e.g., an enzyme from Aspergillus fumigatus AF293 CYP505X. which hydroxylates diclofenac, thereby providing a reference compound. In yet another embodiment, a monooxygenase, such as a flavin monooxygenase, e.g., FMO3, which N- or S-oxygenates nucleophilic nitrogen- or sulfur-containing drugs/chemicals, for instance, amphetamine, benzyldamine, ethionamide, clozapine, deprenyl, tamoxifen, metamphetamine, thiacetazone, and sulindoc sulfide, may be employed.

### REFERENCES

Granström et al., Appl. Microbiol. Biotechnol., 74:277 (2007b).

Granström et al., Appl. Microbiol. Biotechnol., 74:273 (2007a).

Hallborn et al., U.S. Pat. No. 5,866,382.

Iran and Meagher, J. Biosci. Bioeng., 92:585 (2001).

Lee et al., Appl. Environ. Microbiol., 69:6179 (2003).

Oh et al., Biotechnol. Bioeng., 58:440 (1998).

Link et al., J. Bacteriol., 179:6228 (1997).

Wu and Letchworth, Biotechniques, 36:152 (2004).

Handumrongkul et al., Appl. Microbiol. Biotechnol., 49:399 (1998).

Wang et al., Biotechnol. Lett., 29:1409 (2007).

Suzuki et al., J. Biosci. Bioeng., 87:280 (1999).

Häcker et al. Biol. Chem., 380:1395 (1999).

Hou et al., Arch. Biochem. Biophys., 216:296 (1982).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A spray-dried preparation of recombinant microbe comprising a heterologous xylose reductase.

2. The preparation of claim 1 wherein the microbe is a yeast

3. The preparation of claim 2 wherein the yeast is Pichia, Hansenula, Kluyvermyces, Saccharomyces or Candida.

4. The preparation of claim 1 wherein the heterologous xylose reductase is a Neurospora, Pichia, Saccharomyces or Candida xylose reductase.

5. The preparation of claim 1 wherein the microbe is a bacteria.

6. The preparation of claim 1 wherein the recombinant microbe further comprises a recombinant dehydrogenase.

7. The preparation of claim 6 wherein the recombinant dehydrogenase is a bacterial or plant dehydrogenase.

8. A method to prepare a microbial cell biocatalyst preparation, comprising:
a) providing a recombinant microbial cell suspension, or a lysate or crude extract thereof, having a recombinant xylose reductase; and
b) spray drying the microbial cell suspension or a lysate or crude extract thereof, under conditions effective to yield a spray dried preparation suitable for biocatalysis.

9. The method of claim 8 wherein the spray drying includes heating an amount of the cell suspension flowing through an aperature.

10. The method of claim 8 wherein the microbe is a yeast.

11. The method of claim 10 wherein the yeast cell suspension is a Pichia, Hansenula, Kluyvermyces, Candida, or Saccharomyces cell suspension.

12. The method of claim 8 wherein the xylose reductase is a yeast or fungal xylose reductase.

13. The method of claim 8 wherein the xylose reductase is a heterologous xylose reductase.

14. The method of claim 8 wherein the xylose reductase is NADPH-specific.

15. The method of claim 8 wherein the xylose reductase is NADH-specific.

16. The method of claim 8 wherein the xylose reductase is from Neurospora, Pichia, Saccharomyces, or Candida.

17. The method of claim 8 wherein the recombinant cell further comprises a recombinant dehydrogenase.

18. The method of claim 17 wherein the dehydrogenase is a glucose dehydrogenase or formate dehydrogenase.

19. The method of claim 17 wherein the dehydrogenase is from Candida or Bacillus.

20. The method of claim 1 further comprising separating the suspension into a liquid fraction and a solid fraction which contains the microbial cells prior to spray drying.

21. The method of claim 20 further comprising spray drying the separated cells.

22. The method of claim 20 wherein the suspension is separated by centrifugation.

23. The method of claim 20 wherein the suspension is separated using a membrane.

24. A spray-dried microbial cell preparation prepared by the method of claim 8.

25. The preparation of claim 24 wherein the microbe is Pichia, Hansenula, Kluyvermyces, Candida, or Saccharomyces.

26. The preparation of claim 25 wherein the recombinant xylose reductase is a Pichia, Neurospora, Saccharomyces or Candida xylose reductase.

27. A method to prepare xylitol, comprising:
a) providing a spray-dried microbial cell preparation having recombinant xylose reductase; and
b) combining the preparation and a mixture comprising xylose, and optionally NAD⁺ and/or optionally an electron donor, under conditions that yield xylitol.

28. The method of claim 27 wherein the electron donor comprises formate or glucose.

29. The method of claim 27 wherein the preparation further comprises recombinant dehydrogenase.

30. The method of claim 27 wherein the dehydrogenase comprises glucose dehydrogenase or formate dehydrogenase.

31. The method of claim 27 wherein mixture comprises NAD⁺, glucose or formate, or any combination thereof.

32. The method of claim 27 wherein the cell suspension is a Pichia, Hansenula Kluyvermyces, Saccharomycyes or Candida cell suspension.

33. The method of claim 27 further comprising isolating the product.

34. The method of claim 27 wherein the mixture comprises a hemicellulose hydrosylate.
